(19) United States
(12) Patent Application Publication
(10) Pub. No.: US 2020/0370004 A1
(43) Pub. Date: Nov. 26, 2020

(54) HIGH PRODUCTIVITY METHODS FOR GROWING ALGAE

(71) Applicant: Triton Algae Innovations, Inc., San Diego, CA (US)

(72) Inventors: Miller Tran, San Diego, CA (US); Jon Hansen, San Diego, CA (US); John Deaton, San Diego, CA (US); Xun Wang, San Diego, CA (US); Oscar Gonzalez, San Diego, CA (US); Michael Mayfield, San Diego, CA (US); Stephen Mayfield, San Diego, CA (US)

(21) Appl. No.: 16/763,755

(22) PCT Filed: Nov. 13, 2018
(86) PCT No.: PCT/US2018/060830

§ 371 Date: May 13, 2020

Related U.S. Application Data
(60) Provisional application No. 62/587,694, filed on Nov. 17, 2017.
(60) Provisional application No. 62/625,619, filed on Feb. 2, 2018.

(51) Int. Cl. C12N 1/12; C12P 21/00; C12N 15/82; C07K 14/47; A01G 33/00

(52) U.S. Cl. CPC C12N 1/12; C12P 21/00; A01G 33/00; C07K 14/47; C12N 15/8216

(57) ABSTRACT

The present disclosure provides for growing algae with an exogenous organic carbon source as the primary carbon source, in light, dark or limited light conditions. Also provided are expression cassettes for expression of a recombinant protein in an algae species grown in dark or limited light conditions.

### CROSS-REFERENCE TO RELATED APPLICATION(S)

This application claims the benefit of priority under 35 U.S.C. § 119(e) of U.S. Ser. No. 62/587,694, filed Nov. 17, 2017, and from U.S. Ser. No. 62/625,619, filed Feb. 2, 2018, the contents of each of which are incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 13, 2018, is named 20498-202027_SL.txt and is 16 kilobytes in size.

### BACKGROUND OF THE INVENTION

### Field of the Invention

The present disclosure relates to methods for growing algae which provide improved yields, increased efficiencies and reduced costs associated with producing such algae. The present disclosure relates to methods for growing algae in dark and limited light conditions. The present disclosure also relates to methods for growing algae with an exogenous organic carbon source as the primary carbon source.

### Background Information

With a growing population and an increased need for food sources, algae are increasingly being explored as an organism having the potential to supplement existing means of agricultural production. Algae provide an alternative to traditional agriculture and do not require suitable land and climate to propagate. To overcome the hurdles of traditional agriculture, algae can be grown in complete containment thereby eliminating the need for nutrient rich soil or ideal weather. Instead, nutrients can be supplied to the algal cultures and temperatures controlled to ensure optimal growing conditions. By growing algae in complete containment, excess application of nutrients and the harmful runoffs resulting from those nutrients can be avoided.

There are many algae that are grown commercially and used in the human and animal health food chain. However, not all algae meet the requirements necessary to be seriously considered as strains that can be used commercially for human and animal nutrition. Some contributing factors that determine if algae can be used commercially are if the algae are capable of achieving high production titers and if the production process can be done cost effectively. To date, many algae are grown in enclosed fermentation vessels which increases the cost of production both in terms of the large capital investment required and the high energy cost associated with running the fermenters. To overcome these expenses, breakthroughs in production methods that push the biomass titers towards higher densities or that decrease the cost of the inputs are required to achieve economic viability.

One particular genus of algae, Chlamydomonas, has long been studied as a model organism for understanding photosynthesis and other biochemical processes. Chlamydomonas is capable of growing heterotrophically on acetate; however, it lacks the machinery to grow on sugars as a carbon source. This inability to grow on sugars has been documented and repeatedly demonstrated. The cost of acetate is significantly higher than the cost of various sugars (e.g., fructose, sucrose, glucose, galactose), thereby drastically increasing the cost of production of Chlamydomonas. Unlike other genus of green algae, such as Chlorella, Chlamydomonas lack the hexose transporter which facilitates the primary transport of sugars from outside the cell into the cell. Chlamydomonas also lacks a hexose kinases that is localized to the cytosol where it would function to phosphorylate glucose to make glucose-6-phosphate, a key metabolite in the pentose phosphate pathway. Chlamydomonas also lack a cytosol-localized pentose phosphate pathway which converts glucose-6-phosphate into various metabolites which are then used to generate energy for the algae to grow and divide. The industry accepted inability of Chlamydomonas to use sugars as its primary source of carbon is the reason why it is overlooked as an industrial production strain.

Another characteristic of Chlamydomonas is its ability to uptake large quantities of nutrients, beyond what is needed for growth. Although some other algae experience this to some degree, Chlamydomonas is typically much more prolific in its nutrient uptake. This means that conventional media recipes and approaches to designing media based on biomass composition will lead to sub-optimal growth and often inhibitory levels of nutrients. Further, Chlamydomonas is a freshwater algae which is quite different in its nutrient and environmental control requirements compared to the brackish or sea-water strains that do not contain chlorophyll and have been commericaled to date in heterotrophic fermentations, e.g., Crypthecodinium, Schizochytrium, and Thraustochytrium. Compared with other green algae, most wild type strains of Chlamydomonas would naturally produce chlorophyll and hence have a green color, even in the absence of light. Further, often green algae culturing methods typically include some light input at one or more stages, in particular during the inoculum cultivation. Other green algae such as Chlorella have a chitin cell wall and do not require flagella, which can increase robustness in industrial fermentations. Another unique feature of Chlamydomonas is its ability to undergo both sexual and asexual division. All of these reasons lead to a very unique set of challenges which have previously not been overcome with Chlamydomonas through conventional approaches and existing protocols to achieve high performance and attractive composition in reactors.

### SUMMARY

Provided herein are methods for growing algae that improve efficiency, decrease costs and improve yields of biomass and proteins produced by algae. Included herein are methods for growing algae and accumulating protein produced by algae, including recombinant protein, during growth conditions in dark or shaded conditions. Such conditions include growing cells in conditions requiring an exogenous organic carbon source to proliferate. In various embodiments, the methods include administering to the algal culture an exogenous organic carbon source, such as fructose, sucrose, glucose, or acetate. The methods include accumulating protein and/or recombinant protein inside the algal cell or accumulating protein and/or recombinant protein in the media by transporting the protein and/or recombinant protein through a secretory pathway.

Also provided herein are methods that permit green algae, such as Chlamydomonas, to grow on sugar as their primary carbon source. This would significantly decrease the cost of the inputs required to produce algal biomass. Provided herein is an improved method for producing Chlamydomonas which results in a significant decrease in the cost of the inputs necessary to grow the green algae to high densities. Additionally, included herein are methods to modify an existing Chlamydomonas, incapable of growing on sugars as its primary carbon source, to one that is capable of growing on sugars through mating.

Accordingly, in one aspect, the invention provides a method for producing a high-density culture of an algae species. The method includes growing an algae species in the presence of at least one exogenous organic carbon source under aerobic conditions, wherein the algae species is capable of using the organic carbon source as an energy source for growth. In various embodiments, there is a net oxygen consumption and a net CO₂ production. In various embodiments, the the algae species is a Chlamydomonas species, such as Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas maranii, Chlamydomonas proboscigera, and any combination thereof. In various embodiments, the at least one exogenous carbon source is selected from the group consisting of glucose, fructose, sucrose, maltose, glycerol, molasses, starch, cellulose, acetate, and any combination thereof.

In various embodiments, the Chlamydomonas species is grown in the presence of light, in limited light conditions, or in the dark. In various embodiments, the Chlamydomonas species is grown to a density of at least 30 g/L, at least 35 g/L, at least 40 g/L, at least 45 g/L, at least 50 g/L, at least 55 g/L, at least 60 g/L, at least 65 g/L, at least 70 g/L, at least 75 g/L, at least 80 g/L, at least 85 g/L, at least 90 g/L, at least 95 g/L, at least 100 g/L, at least 105 g/L, at least 110 g/L, at least 115 g/L, at least 120 g/L, or at least 125 g/L. In various embodiments, the culture is grown in a high density fermenter. In various embodiments, exogenous air or oxygen is supplied during the growing step.

In another aspect, the invention provides a method for accumulating a recombinant protein from a culture of a Chlamydomonas species. The method includes providing one or more cells of a recombinant Chlamydomonas species capable of expressing a recombinant protein, growing the one or more cells in the presence of at least one exogenous organic carbon source under aerobic conditions to generate a culture of the recombinant Chlamydomonas species, wherein the Chlamydomonas species uses the organic carbon source as an energy source for growth, and harvesting the recombinant protein from the culture. In various embodiments, there is a net oxygen consumption and a net CO₂ production. In various embodiments, the the algae species is a Chlamydomonas species, such as Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas maranii, Chlamydomonas proboscigera, and any combination thereof. In various embodiments, the at least one exogenous carbon source is selected from the group consisting of glucose, fructose, sucrose, maltose, glycerol, molasses, starch, cellulose, acetate, and any combination thereof.

In various embodiments, the Chlamydomonas species is grown in the presence of light, in limited light conditions, or in the dark. In various embodiments, the Chlamydomonas species is grown to a density of at least 30 g/L, at least 35 g/L, at least 40 g/L, at least 45 g/L, at least 50 g/L, at least 55 g/L, at least 60 g/L, at least 65 g/L, at least 70 g/L, at least 75 g/L, at least 80 g/L, at least 85 g/L, at least 90 g/L, at least 95 g/L, at least 100 g/L, at least 105 g/L, at least 110 g/L, at least 115 g/L, at least 120 g/L, or at least 125 g/L. In various embodiments, the culture includes liquid media and cells, and the recombinant protein is harvested from the liquid media, from the cells of the culture, or both.

In various embodiments of any of the methods described herein, the recombinant protein is expressed in a chloroplast. In various embodiments, expression of a recombinant gene of interest is driven using the 16S promoter of the endogenous chloroplast genome. In various embodiments, productivity of Chlamydomonas cultivation in grams (g) of Chlamydomonas biomass per liter (L) of culture is at least about 0.3 g/L/hour, at least about 0.5 g/L/hour, at least about 0.6 g/L/hour, at least about 0.9 g/L/hour, at least about 1.5 g/L/hour, or at least about 2 g/L/hour. In various embodiments, conversion efficiency of Chlamydomonas biomass on the exogenous organic carbon source is at least about 0.3 g biomass/g carbon source, at least about 0.4 g biomass/g carbon source, at least about 0.5 g biomass/g carbon source, at least about 0.6 g biomass/g carbon source, or at least about 0.7 g biomass/g carbon source. In various embodiments, total protein content of Chlamydomonas biomass of the Chlamydomonas culture is at least about 20%, at least about 30%, at least about 40%, at least about 45%, at least about 50%, at least about 55%, at least about 60%, or at least about 70%. In various embodiments, the Chlamydomonas culture at time of harvest has a productivity rate of at least about 0.3 g biomass per liter per hour and a density of 50 g biomass per liter of culture.

In another aspect, the present invention provides an expression cassette. The expression cassette includes an algae 16S promoter fused to a 5′-untranslated region (5′ UTR) and a nucleic acid molecule encoding a recombinant protein, wherein the 5′UTR is selected from the group consisting of psbM, psaA, psaB, psbI, psbK, clpP, rpl14, rps7, rps14, and rps19 5′UTR. In various embodiments, the expression cassette provides expression of the recombinant protein in an algae species, such as a Chlamydomonas species, grown in dark or limited light conditions. In various embodiments, the 5′UTR includes a sequence selected from the group consisting of SEQ ID NOs:12-20 and 21, or includes a sequence with at least 80% sequence identity to a sequence selected from the group consisting of SEQ ID NOs: 12-20 and 21. In various embodiments, the 16S promoter is a 16S promoter from a Chlamydomonas species. In various embodiments, the 16S promoter is SEQ ID NO: 1 or a sequence with at least 80% sequence identity to SEQ ID NO: 1. In various embodiments, the expression cassette includes a sequence selected from the group consisting of SEQ ID NOs: 2-10 and 11, or includes a sequence with at least 80% sequence identity to a sequence selected from the group consisting of SEQ ID NOs: 2-10 and 11.

In another aspect, the invention provides a method of expressing a recombinant protein in an algae. The method includes introducing an expression cassette into an algae, wherein the expression cassette comprises an algae 16S promoter fused to a 5′-untranslated region (5′ UTR) and a nucleic acid molecule encoding a recombinant protein, and growing the algae under dark or limited light conditions, wherein the 5′UTR is selected from the group consisting of psbM, psaA, psaB, psbI, psbK, clpP, rpl14, rps7, rps14, and rps19 5′UTR. In various embodiments, the the algae species is a Chlamydomonas species, such as Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas maranii, Chlamydomonas proboscigera, and any combination thereof.

In various embodiments, the 5′UTR includes a sequence selected from the group consisting of SEQ ID NOs:12-20 and 21, or includes a sequence with at least 80% sequence identity to a sequence selected from the group consisting of SEQ ID NOs: 12-20 and 21. In various embodiments, the 16S promoter is a 16S promoter from a Chlamydomonas species. In various embodiments, the 16S promoter is SEQ ID NO: 1 or a sequence with at least 80% sequence identity to SEQ ID NO: 1. In various embodiments, the expression cassette includes a sequence selected from the group consisting of SEQ ID NOs: 2-10 and 11, or includes a sequence with at least 80% sequence identity to a sequence selected from the group consisting of SEQ ID NOs: 2-10 and 11.

### BRIEF DESCRIPTION OF THE DRAWINGS

These and other features, aspects, and advantages of the claimed invention will become better understood with regard to the following description, appended claims and accompanying figures where:

FIG. 1 is a pictorial diagram showing an exemplary molecular construct used to achieve expression of recombinant proteins in dark or limited light conditions.

FIG. 2 is a depiction of the DNA sequence (SEQ ID NO: 7) of the synthetic fusions of the 16S promoter (SEQ ID NO: 1) and psbM 5′ untranslated region (SEQ ID NO: 17) used to drive protein accumulation in dark or shaded conditions.

FIG. 3 is a depiction of the DNA sequence (SEQ ID NO: 2) of the synthetic fusions of the 16S promoter (SEQ ID NO: 1) and psaA 5′ untranslated region (SEQ ID NO: 12) used to drive protein accumulation in dark or shaded conditions.

FIG. 4 is a depiction of the DNA sequence (SEQ ID NO: 3) of the synthetic fusions of the 16S promoter (SEQ ID NO: 1) and psaB 5′ untranslated region (SEQ ID NO: 13) used to drive protein accumulation in dark or shaded conditions.

FIG. 5 is a depiction of the DNA sequence (SEQ ID NO. 5) of the synthetic fusions of the 16S promoter (SEQ ID NO: 1) and psbI 5′ untranslated region (SEQ ID NO: 15) used to drive protein accumulation in dark or shaded conditions.

FIG. 6 is a depiction of the DNA sequence (SEQ ID NO: 6) of the synthetic fusions of the 16S promoter (SEQ ID NO: 1) and psbK 5′ (SEQ ID NO: 16) untranslated region used to drive protein accumulation in dark or shaded conditions.

FIG. 7 is a depiction of the DNA sequence (SEQ ID NO: 8) of the synthetic fusions of the 16S promoter (SEQ ID NO: 1) and rpl14 5′ untranslated region (SEQ ID NO: 20) used to drive protein accumulation in dark or shaded conditions.

FIG. 8 is a depiction of the DNA sequence (SEQ ID NO: 4) of the synthetic fusions of the 16S promoter (SEQ ID NO: 1) and clpP 5′ untranslated region (SEQ ID NO: 14) used to drive protein accumulation in dark or shaded conditions.

FIG. 9 is a depiction of the DNA sequence (SEQ ID NO: 9) of the synthetic fusions of the 16S promoter (SEQ ID NO: 1) and rps7 5′ untranslated region (SEQ ID NO: 19) used to drive protein accumulation in dark or shaded conditions.

FIG. 10 is a depiction of the DNA sequence (SEQ ID NO: 10) of the synthetic fusions of the 16S promoter (SEQ ID NO: 1) and rps14 5′ untranslated region (SEQ ID NO: 20) used to drive protein accumulation in dark or shaded conditions.

FIG. 11 is a depiction of the DNA sequence (SEQ ID NO: 11) of the synthetic fusions of the 16S promoter (SEQ ID NO: 1) and rps19 5′ untranslated region (SEQ ID NO: 21) used to drive protein accumulation in dark or shaded conditions.

FIG. 12 is pictorial diagram showing results from a western blot and ELISA demonstrating accumulation of a recombinant flag tagged protein under dark conditions while under genetic control of the 16S promoter and the various 5′ untranslated region.

FIG. 13 is a graphical diagram showing accumulation of osteopontin protein over time of an algae culture transformed with the 16S promoter and psbM 5′UTR driving the expression of recombinant bovine osteopontin.

FIG. 14 is a pictorial diagram showing growth of Chlamydomonas strains on exogenous organic carbon sources.

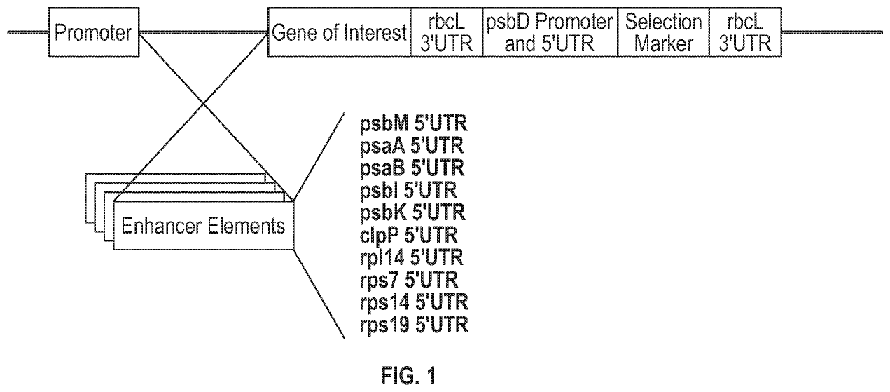

### DETAILED DESCRIPTION

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps, as well as embodiments in which those elements or steps are included and may also include additional elements or steps.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Algae, as used herein, refer to non-vascular algae and may include organisms classified as microalgae. It should be noted that in the present disclosure the terms microalgae and algae are used interchangeably. Non-limiting examples of genera of microalgae that may be used to practice the methods disclosed herein include Prochlorophyta, Rhodophyta, Chlorophyta, Heterokontophyta, Tribophyta, Glaucophyta, Chlorarachniophytes, Euglenophyta, Euglenoids, Haptophyta, Chrysophyta, Cryptophyta, Cryptomonads, Dinophyta, Dinoflagellata, Pyrmnesiophyta, Bacillariophyta, Xanthophyta, Eustigmatophyta, Raphidophyta and Phaeophyta. In various embodiments, the algae used to practice the methods described herein is of the genus Chlamydomonas. In various embodiments, the algae used in practicing the disclosed methods are Chlamydomonas reinhardtii (C. reinhardtii).

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges can independently be included or excluded in the range, and each range where either, neither or both limits are included in the smaller ranges is also encompassed, subject to any specifically excluded limit in the stated range. Where the stated range includes one or both of the limits, ranges excluding either or both of those included limits are also included.

The term “dark” or shaded” means conditions that are <150 microeinsteins.

“Light conditions” means a condition where there is a net O₂ production and CO₂ production.

The term “limited light” means conditions where there is a net positive carbon dioxide (CO₂) production and oxygen (O₂) evolution by the algae culture.

“Phototrophic” or “photoautotrophic algae” refers to algae that use photon capture as a source of energy and can fix inorganic carbon. As such phototrophic algae are capable of using inorganic carbon in the presence of light as a source of metabolic carbon.

As used herein, “heterotrophic algae” refers to algae that do not use photon capture as an energy source, but instead rely on organic carbon sources.

“Mixotrophic algae” means those algae that are capable of using photon capture and inorganic carbon fixation to support growth, but in the absence of light may use organic carbon as an energy source. Thus, mixotrophic algae have the metabolic characteristics of both phototrophic and heterotrophic algae.

Sugar, unless otherwise specified, includes all monosaccharides, disaccharides, oligosaccharides and polysaccharides. Non-limiting examples of monosaccharides are fructose, glucose and galactose. Non-limiting examples of disaccharides are lactose, maltose, and sucrose. Non-limiting examples oligosaccharides are fructo-oligosaccharides and galactooligosaccharides.

As used herein, an “expression cassette” refers to a portion of DNA that includes one or more genes and one or more regulatory sequences controlling their expression. In each successful transformation, the expression cassette directs the cell's machinery to make RNA and/or protein(s) encoded by the one or more genes.

As used herein, the term “gene” means the deoxyribonucleotide sequences that codes for a molecule that has a function. A “structural gene” refers to a gene that codes for an RNA or protein other than a regulatory factor, but is nonetheless encompassed within the definition of “gene.” A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences (or alternatively, 5′ untranslated regions (5′ UTRs)). The sequences which are located 3′ or downstream of the coding region and which are present on the mRNA are referred to as 3′ non-translated sequences. The term “gene” encompasses both cDNA and genomic forms of a gene.

### Growth Conditions and Methods for Algae Production

Provided herein are methods for accumulating protein in algae. In some embodiments, the protein accumulated is one or more naturally occurring proteins. In some embodiments, the protein accumulated is a heterologous protein, such as a recombinant protein. In some embodiments, the accumulated protein is accumulated intracellularly. In some embodiments, the protein is accumulated in the culture media in which the algae are grown. In some embodiments of the methods for accumulating protein, the algae are grown in dark heterotrophic conditions. In some embodiments of the methods for accumulating protein, the algae are grown in limited light mixotrophic conditions. These methods include genetic tools and production processes that facilitate the accumulation of proteins without the requirement of light illumination on the algal cells. Also provided herein are methods for growing algae to high density and for accumulating protein expressed by algae under conditions of aerobic heterotrophic cultivation.

Non-limiting examples of genera of microalgae that may be used to practice the methods disclosed herein include Prochlorophyta, Rhodophyta, Chlorophyta, Heterokontophyta, Tribophyta, Glaucophyta, Chlorarachniophytes, Euglenophyta, Euglenoids, Haptophyta, Chrysophyta, Cryptophyta, Cryptomonads, Dinophyta, Dinoflagellata, Pyrmnesiophyta, Bacillariophyta, Xanthophyta, Eustigmatophyta, Raphidophyta and Phaeophyta. In some embodiments, the algae used to practice the methods described herein is of the genus Chlamydomonas. Exemplary Chlamydomonas species for use with the methods herein include, but are not limited to, Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas marvanii, Chlamydomonas pseudococum, Chlamydomonas pseudoglou, Chlamydomonas sno, or Chlamydomonas proboscigera. In some embodiments, the algae used in practicing the disclosed methods is Chlamydomonas reinhardtii (C. reinhardtii).

In some embodiments, mating is employed to create strains of algae, including but not limited to strains of Chlamydomonas for use with the methods herein. Mating can be accomplished by genetically crossing two mating types, such as a mating type minus and a mating type positive strain of Chlamydomonas. In a non-limiting example of mating, the mating type minus strain of Chlamydomonas donates its mitochondrial genome to daughter cells and the mating type positive strain donates its chloroplast plastid genome to the same daughter cells. Cells of the Chlamydomonas are nitrogen starved to stimulate sexual reproduction and the Chlamydomonas species form a zygote after the step of mating. Unmated Chlamydomonas can be removed by exposure to chloroform which selectively kills the unmated cells. The zygotes can then be repropagated by addition of nitrogen repleate media. In some instances, the Chlamydomonas being mated have flagella prior to formation of zygotes. Other methods for mating algae are available in the art and can be employed with the methods described herein.

### Growth in Dark and Limited Light Conditions

In some embodiments of the methods herein, the algae are grown under conditions which do not permit photosynthesis, (e.g., the organism may be grown in the absence of light). In some embodiments, the algae are grown in “dark” or shaded” conditions that are <150 microeinsteins. In some embodiments, algae used in the practice of the present disclosure may be mixotrophic or heterotrophic.

In growth conditions where a microorganism is not capable of photosynthesis (naturally or due to selection), the methods include providing the algae with the necessary nutrients to support growth in the absence of light and photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or any organism-specific requirement. Organic carbon sources include any source of carbon which the host organism is able to metabolize including, but not limited to, acetate, simple carbohydrates (e.g., glucose, sucrose, lactose), complex carbohydrates (e.g., starch, glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

In various embodiments, the algae are grown in the absence of light. Exemplary methods for the production of high density algae cultures in the absence of light, can be found in PCT/US2017/046831, published as WO201838960, entitled IMPROVED METHOD FOR GROWING ALGAE, which is incorporated herein by reference in its entirety.

In some embodiments of the methods herein, protein accumulates inside the algae cell. This accumulation can occur in chloroplasts, mitochondria, cytosol, the endoplasmic reticulum or the periplasmic space. In some embodiments, the protein accumulated in such organelles or cellular spaces is one or more recombinant proteins. In some embodiments of the methods herein, protein is accumulated outside of the cells in the culture media. In some embodiments, the protein accumulated in the culture media is one or more recombinant proteins.

In some embodiments, the recombinant protein accumulates in the algal cell and is from about 0.01% of the whole cell to about 20% of the whole cell by weight. In other embodiments, the recombinant protein comprises about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.1%, or about 0.01% of the weight of the algal culture. In some embodiments, the recombinant protein accumulates outside the cell and is from about 0.01% of the whole cell to about 20% of the whole cell by weight. In other embodiments, the recombinant protein accumulates in the media as about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.1%, or about 0.01% of the weight of the algal culture.

In some embodiments, the methods herein provide a high density and high productivity culture of algae. In some embodiments, the productivity of the culture in grams (g) of algae biomass per liter (L) of culture is at least about 0.3 g/L/hour, at least about 0.5 g/L/hour, at least about 0.6 g/L/hour, at least about 0.9 g/L/hour, at least about 1.5 g/L/hour, or at least about 2 g/L/hour. In some embodiments, the conversion efficiency of the exogenous organic carbon source provided to biomass of algae is at least about 0.3 g biomass/g carbon source, at least about 0.4 g biomass/g carbon source, at least about 0.5 g biomass/g carbon source, at least about 0.6 g biomass/g carbon source, or at least about 0.7 g biomass/g carbon source. In some embodiments, the algae grown in the presence of the exogenous organic carbon source produce a high protein algae biomass. In some embodiments, the high protein biomass is at least about 20%, at least about 30%, at least about 40%, at least about 45%, at least about 50%, at least about 55%, at least about 60%, or at least about 70% protein per total biomass weight.

In some embodiments, the methods herein produce an algae biomass with a desired amino acid content (expressed as amino acid fraction per total protein content). In some embodiments, the algae biomass has a lysine fraction of at least about 5% of the total protein content. In some embodiments, the algae biomass has a methionine fraction of at least about 2% of the total protein content. In some embodiments, the algae biomass has a threonine fraction of at least about 4% of the total protein content. In some embodiments, the algae biomass has a tryptophan fraction of at least about 2% of the total protein content. In some embodiments, the algae biomass has a valine fraction of at least about 5% of the total protein content.

In some embodiments, the methods herein include growing a production culture of algae in defined pH and/or defined temperature conditions. In some embodiments, the production culture is aerobically at a pH of between about 2.0 and 10.0. In some embodiments, the pH of the production culture is maintained at about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In some embodiments, the production culture is grown at a temperature between about 5° C. to about 50° C. In some embodiments, the temperature is between about 5° C. to about 10° C. about 10° C. to about 15° C., about 15° C. to about 20° C., about 20° C. to about 25° C., about 25° C. to about 30° C., about 30° C. to about 35° C., about 30° C. to about 40° C., about 35° C. to about 40° C., about 40° C. to about 45° C., or about 45° C. to about 50° C. Thus, in some embodiments, the temperature is or is about 30° C., 31° C., 32° C., 33° C., 34° C., 35° C., 36° C., 37° C., 38° C., 39° C., or 40° C.

### Carbon Sources

Also provided herein are methods for growing algae where one or more exogenous organic carbon sources are provided to the algae culture for use as an energy source and/or a source of carbon. Such exogenous organic carbon sources that may be provided with the methods herein include, but are not limited to, glucose, fructose, sucrose, maltose, glycerol, molasses, starch, cellulose, acetate, and any combination thereof. In some embodiments, the methods include an aerobic, heterotrophic cultivation of a high density culture of algae wherein the cultures are grown using an exogenous organic carbon source or a combination of exogenous organic carbon sources. In some embodiments, the methods include an aerobic, heterotrophic cultivation of a high density culture of algae wherein the cultures are grown using a sugar or a combination of sugars as an exogenous organic carbon source. In some embodiments, a combination of other exogenous carbon sources, such as glycerol and acetate, or a combination of sugar and non-sugar exogenous organic carbon sources is employed in the methods.

In some embodiments the algae are grown using one or more exogenous carbon sources to achieve a target density of between about 10 g/L and about 300 g/L dry cell weight. In certain embodiments, the culture achieves a target density of at least about 10 g/L, at least about 25 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L or at least about 200 g/L dry cell weight. In other embodiments, the target density is between about 50 g/L and about 75 g/L, between about 75 g/L and about 100 g/L, between about 100 g/L and about 125 g/L, between about 125 g/L and about 150 g/L, between about 150 g/L and about 175 g/L or between about 175 g/L and about 200 g/L dry cell weight. In certain embodiments the production culture is grown to a density of about 25 g/L, about 30 g/L, about 35 g/L, about 40 g/L, about 45 g/L, about 50 g/L, about 55 g/L, about 65 g/L, about 70 g/L, about 75 g/L, about 80 g/L, about 85 g/L, about 90 g/L, about 95 g/L, about 100 g/L, about 105 g/L, about 110 g/L, about 115 g/L, about 120 g/L, about 125 g/L, about 130 g/L, about 135 g/L, about 140 g/L, about 145 g/L, about 150 g/L, about 155 g/L, about 160 g/L, about 165 g/L, about 170 g/L, about 175 g/L, about 180 g/L, about 185 g/L, about 190 g/L, about 195 g/L or about 200 g/L dry cell weight before harvesting. In some embodiments, the target density or concentration is reached within about 96 hours after the start of the production culture. In some embodiments, the target density or concentration is reached within about 96 hours, about 120 hours, about 150 hours, about 175 hours, about 200 hours, about 220 hours, or about 250 hours after the start of the production culture. In some embodiments, the target density or concentration is reached within about 250 hours after the start of the production culture.

In some embodiments, the algae grown with the methods described herein is a Chlamydomonas species. The Chlamydomonas sp. used in the methods of growing with an exogenous carbon source can be any species that is capable of heterotrophic or mixotrophic growth an exogenous organic carbon source or any species that is capable of mating with a Chlamydomonas with such growth ability such that the resulting strain inherits the ability to grow on the exogenous organic carbon source. In some embodiments, the species selected has the ability to grow on one or more sugars as a carbon source. Exemplary Chlamydomonas species for use with the methods herein include, but are not limited to, Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas maranii, Chlamydomonas pseudococum, Chlamydomonas pseudoglou, Chlamydomonas sno, or Chlamydomonas proboscigera.

In some embodiments, the Chlamydomonas sp. grown on the one or more sugars is a wild-type species that does not contain a heterologous or exogenous gene. In other embodiments, the Chlamydomonas sp. grown on the one or more sugars is recombinant and/or contains at least one heterologous or exogenous gene. In some embodiments, the heterologous or exogenous gene is from a species other than Chlamydomonas. In other embodiments, the heterologous or exogenous gene is from Chlamydomonas species.

In some embodiments, the Chlamydomonas sp. used in the method of growing on one or more organic exogenous carbon sources, such as one or more sugars, is derived from a Chlamydomonas which was previously incapable of growing on an organic carbon source as its primary carbon source, and the methods herein include providing such capability whereby the Chlamydomonas sp. has inherited the machinery to grow on an organic carbon source, such as a sugar, as its primary carbon source through a mating, breeding, cross, or protoplast fusion with another strain of algae.

In various embodiments, the organic carbon source, such as a sugar, that is being consumed by the algae as an exogenous organic carbon source is found in the base media. In various embodiments, the organic carbon source, such as a sugar, that is being consumed by the algae as a carbon source is being supplied in the feed media.

### Light Conditions

In some embodiments of the methods herein herein, the algae are grown in conditions that are light-limited and the algae culture has a net positive CO₂ production and O₂ evolution. In various embodiments, the production culture is grown under light-limited conditions where the exogenous organic carbon source used for energy is an organic carbon source such as glucose, fructose, sucrose, maltose, glycerol, molasses, starch, cellulose, acetate, and any combination thereof.

In various embodiments, the production culture is grown in light-limited conditions where the exogenous organic carbon source used for energy is something other than sugar, such as acetate or glycerol. In some embodiments, the algal culture grown in limited light conditions is a Chlamydomonas species. In some embodiments, the algal culture grown in limited light conditions is Chlamydomonas reinhardtii.

In various embodiments, the production culture is grown in light conditions where sugars are still being consumed and metabolized by the algae culture and there is a net O₂ production and CO₂ production. In various embodiments, the production culture is grown under light conditions where the exogenous organic carbon source used for energy is a sugar. In various embodiments, the production culture is grown in light conditions where the exogenous organic carbon source used for energy is something other than sugar, such as acetate or glycerol. In various embodiments, the production culture is grown in light conditions where the exogenous organic carbon source used for energy is a combination of sugar and non-sugar carbon sources. In some embodiments, the algal culture grown in such light conditions is a Chlamydomonas species. In some embodiments, the algal culture grown in such light conditions is a Chlamydomonas reinhardtii.

In various embodiments, the algal production culture is grown in the dark where the exogenous carbon source, such as one or more sugars, is the only carbon source that is used to generate metabolic energy. In various embodiments, the production culture is grown in the dark where the exogenous organic carbon source used for energy is a sugar. In various embodiments, the production culture is grown in the dark where the exogenous organic carbon source used for energy is something other than sugar, such as acetate or glycerol. In various embodiments, the production culture is grown in the dark where the exogenous organic carbon source used for energy is a combination of sugar and non-sugar carbon sources. In some embodiments, the algal culture grown in the dark is a Chlamydomonas species. In some embodiments, the algal culture grown in the dark is a Chlamydomonas reinhardtii.

In some embodiments, the algal culture is grown mixotrophically, where there is active photosynthesis and consumption of an exogenous carbon source. In various embodiments, the production culture is grown mixotrophically where the exogenous organic carbon source used for energy is a sugar. In various embodiments, the production culture is grown mixotrophically where the exogenous organic carbon source used for energy is something other than sugar, such as acetate or glycerol. In various embodiments, the production culture is grown mixotrophically where the exogenous organic carbon source used for energy is a combination of sugar and non-sugar carbon sources. In some embodiments, the algal culture grown mixotrophically is a Chlamydomonas species. In some embodiments, the algal culture grown mixotrophically is a Chlamydomonas reinhardtii.

In some embodiments of the methods herein, there is provided a culture of one or more species of Chlamydomonas algae under growth conditions of dark, limited light, light conditions and/or with one or more exogenous carbon sources, where the density of the culture increases at a rate of between about 50% and about 3000%, between about 50% and about 100%, between about 100% and about 150%, between about 150% and about 200%, between about 200% and about 250% or between about 250% and about 300% per 24 hour period.

In another aspect, there is provided a culture of one or more species of Chlamydomonas algae able to be cultured under growth conditions of dark, limited light, light conditions and/or with one or more carbon sources, where the density of the culture increases at least about 50%, at least about 75%, at least about 100%, at least about 125%, at least about 150%, at least about 175%, at least about 200%, at least about 225%, at least about 250%, at least about 275%, or at least about 300% per 24 hour period.

Also provided is an algal culture of one or more species of Chlamydomonas algae able to be cultured under steady state conditions where the culture has a density of algae of at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, or at least about 200 g/L dry cell weight, where steady state is defined as a state where the concentration of algae in the culture is increasing between about about 0.1% and about 500% per 24 hour period.

In some embodiments of the methods herein, the algae so cultured produces chlorophyll. In some embodiments, the chlorophyll content of the algae during production is at least about 1%, at least about 2%, at least about 5%, at least about 10%, or at least about 20%.

### Culturing Methods

As disclosed herein, the methods for culturing algae can include providing conditions which improve the efficiency, health and/or production properties of the culture. Such conditions include monitoring and/or modulating nutrient content, pH, light exposure, density and other features of the culture.

In some embodiments, the algae culture is provided with an exogenous carbon organic source. In some embodiments, the exogenous carbon source is provided to the algae culture at a fixed ratio to nitrogen feed. In various embodiments, the nitrogen feed can be adjusted to maintain a fixed pH.

In some embodiments, the exogenous organic carbon source is provided throughout the fermentation (production) period for the algae culture. In some embodiments, the exogenous organic carbon source is provided during a portion of fermentation period. In some embodiments, the exogenous organic carbon source is added in response to changes in dissolved oxygen concentration in the culture media. In some embodiments, the exogenous organic carbon source is added to maintain a respiratory quotient of between about 0.9 and about 1.1. In some embodiments, dissolved oxygen concentration in the culture media is maintained at below about 1%, below about 3%, or below about 5% during fermentation after the biomass reaches a density of at least about 20 g/L, at least about 30 g/L, at least about 40 g/L or at least about 50 g/L.

Adjustments in the provision of nutrients, exogenous organic carbon source, minerals, and/or oxygen to the culture can be made in response to real time measurements of concentrations in the culture, such as by on-line measurements in a bioreactor. Such adjustments also can be made in response to off-line measurements of concentrations from the culture.

Exemplary conditions for culturing algae, such as culturing a Chlamydomonas species, include starting a production (fermentation) culture at a biomass density of at least about 0.5 g/L. At the start of fermentation, the ratio of total broth (culture media) conductivity/density of cell culture is below about 1, below about 5, below about 10, below about 15, or below about 20 mS/cm/mL to g/L of cell culture. In some instances, the total broth conductivity is maintained at below about 5 mS/cm/ml, below about 10 mS/cm/ml, below about 15 mS/cm/ml, or below about 20 mS/cm/ml throughout fermentation. In some embodiments, dissolved oxygen is maintained at below about 1%, below about 3% or below about 5% during fermentation after the biomass reaches at least about 20 g/L, at least about 30 g/L, at least about 40 g/L or at least about 50 g/L.

In some embodiments, a semi-continuous mode of operation is employed such that during fermentation some culture may remain in the fermentor after a portion is removed or harvested, and fresh media can then be added to start a new fermentation. In some embodiments of semi-continuous mode, up to about 5%, about 10%, about 15%, about 20%, about 30%, about 40%, about 50%, about 60%, about 70%, about 80%, or about 90% broth is left in the fermentor with fresh media added or fed to start a subsequent fermentation. In some embodiments, a continuous mode of operation is employed such that during fermentation broth (culture media) is fed into the reactor as broth (with cells) is being harvested.

In some embodiments, algae is cultured under aerobic conditions in the presence of an exogenous organic carbon source to produce a high-density culture of a Chlamydomonas species with the resulting culture having a net oxygen consumption and CO₂ production. In some instances, the net oxygen consumption and CO₂ production occurs where the total biomass density is at least about 60 g/L.

### Expression Cassettes

In another aspect, the present disclosure provides expression cassettes that allow a gene of interest to be expressed in algae grown in the dark or light limited conditions. In various embodiments, the expression cassettes of the invention can include a nucleic acid sequence encoding a protein of interest in a form suitable for expression of the nucleic acid molecule in a host cell (i.e., an algal cell), which means that the expression cassettes include one or more regulatory elements, which may be selected on the basis of the algal cells to be used for expression, that is operatively-linked to the nucleic acid sequence to be expressed. As used herein, “operably linked” is intended to mean that the nucleotide sequence of interest is linked to the regulatory element(s) in a manner that allows for expression (e.g., transcription and translation) of the nucleotide sequence in a host cell when the vector is introduced into the host cell.

The term “regulatory element” is intended to include promoters, enhancers, internal ribosomal entry sites (IRES), and other expression control elements. Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cells and those that direct expression of the nucleotide sequence only in certain host cells (e.g., cell-specific regulatory sequences).

As used herein, a “promoter” is defined as a regulatory DNA sequence generally located upstream of a gene that mediates the initiation of transcription by directing RNA polymerase to bind to DNA and initiating RNA synthesis. A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular compound or protein), it may be a spatially restricted promoter (i.e., transcriptional control element, enhancer, etc.) (e.g., tissue specific promoter, cell type specific promoter, etc.), and it may be a temporally restricted promoter (i.e., the promoter is in the “ON” state or “OFF” state during specific stages of embryonic development or during specific stages of a biological process. An exemplary regulatory element useful in the expression cassettes of the invention is an algae 16S promoter.

As used herein, “5′ untranslated region” or “5′-UTR” (also known as a leader sequence or leader RNA) refers to a region of mRNA that is directly upstream from the initiation codon and important for the regulation of translation of a transcript. While called untranslated, the 5′ UTR or a portion of it is sometimes translated into a protein product. This product can then regulate the translation of the main coding sequence of the mRNA. As used herein, “3′ untranslated region” or “3′-UTR” refers to the section of messenger RNA (mRNA) that immediately follows the translation termination codon. An mRNA molecule is transcribed from the DNA sequence and is later translated into protein

Accordingly, the invention provides expression cassettes comprising an algae 16S promoter fused to a 5′-untranslated region (5′ UTR) and a nucleic acid molecule encoding a recombinant protein of interest, wherein the 5′UTR is selected from the group consisting of psbM, psaA, psaB, psbI, psbK, clpP, rpl4, rps7, rps14, and rps19 5′UTR. Such expression cassettes may be introduced into an algae (i.e., algal cell) such that when grown under dark or limited light conditions, the algae expresses the recombinant protein of interest.

### Recombinant and Exogenous (Heterologous) Protein Production

As provided herein, the methods for growth of algae in conditions of dark, limited light, light conditions and/or with one or more exogenous organic carbon sources can be used for producing a heterologous protein. In some embodiments, the heterologous protein is produced from the expression of a non-native exogenous gene. In some embodiments, the heterologous protein is a recombinant protein, such as produced from nucleic acid introduced into the algae through recombinant nucleic acid technology available in the art.

In some embodiments, heterologous protein is produced by inoculating growth media with a substantially pure culture of at least one Chlamydomonas sp. expressing at least one non-native exogenous gene. The method includes inoculating a production culture with an inoculum comprising the substantially pure culture containing about about 0.01 to about 250 g/L of at least one Chlamydomonas sp. expressing at least one non-natural exogenous gene. Non-limiting examples of heterologous proteins that can be produced by the methods herein include therapeutic proteins, vaccines, nutritional proteins, enzymes, antibodies, milk proteins, iron-binding and heme-binding proteins.

In some embodiments, the production culture to produce the heterologous protein is grown aerobically at a pH between about 2.0 and about 10.0. In some embodiments, the pH of the production culture is maintained at about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5 or about 10.0. In some embodiments, the pH is monitored such that at a certain pH (set point), the provision of exogenous organic carbon source provided to the culture is commenced or stopped. In some embodiments, the provision of exogenous organic carbon source commences when the pH exceeds about 7.5 and the provision of exogenous organic carbon source is discontinued after the pH decreases below about 6.8.

In some embodiments, the production culture is grown at a temperature of between about 5° C. to about 50° C. In some embodiments, the temperature is between about 5° C. to about 10° C., about 10° C. to about 15° C., about 15° C. to about 20° C., about 20° C. to about 25° C., about 25° C. to about 30° C., about 30° C. to about 35° C., about 30° C. to about 40° C., about 35° C. to about 40° C., about 40° C. to about 45° C., or about 45° C. to about 50° C. In some embodiments, the temperature is or is about 30° C., 31° C., 32° C., 33° C., 34° C., 35° C. 36° C., 37° C., 38° C., 39° C., or 40° C.

In various embodiments, the production culture expressing a heterologous protein is grown in conditions that are light-limited. In various embodiments, the production culture expressing a heterologous protein is grown under light-limited conditions where the exogenous organic carbon source used for energy is a sugar. In various embodiments, the production culture expressing a heterologous protein is grown in light-limited conditions, where the exogenous organic carbon source used for energy is something other than sugar such as acetate or glycerol. In various embodiments, the production culture expressing a heterologous protein is grown in light conditions where sugars are still being consumed and metabolized by the algae culture. In various embodiments, the production culture expressing a heterologous protein is grown under light conditions, where the exogenous organic carbon source used for energy is a sugar. In various embodiments, the production culture expressing a heterologous protein is grown in light conditions, where the exogenous organic carbon source used for energy is something other than sugar such as acetate or glycerol. In various embodiments, the algal production culture expressing a heterologous protein is grown in the dark, where the sugar is the only carbon source that is used to generate metabolic energy. In various embodiments, the production culture expressing a heterologous protein is grown in the dark, where the exogenous organic carbon source used for energy is a sugar. In various embodiments, the production culture expressing a heterologous protein is grown in the dark, where the exogenous organic carbon source used for energy is something other than sugar such as acetate or glycerol. In various embodiments, the algal culture expressing a heterologous protein is grown mixotrophically. In various embodiments, the production culture expressing a heterologous protein is grown mixotrophically, where the exogenous organic carbon source used for energy is a sugar. In various embodiments, the production culture expressing a heterologous protein is grown mixotrophically, where the exogenous organic carbon source used for energy is something other than sugar such as acetate or glycerol.

In some embodiments, the target concentration for production of the heterologous protein is at least about 65 g/L or at least about 70 g/L. In other embodiments, the target concentration is at least about 75 g/L, at least about 80 g/L, at least about 90 g/L, at least about 95 g/L, at least about 100 g/L, at least about 105 g/L, at least about 110 g/L, at least about 115 g/L, at least about 120 g/L, at least about 125 g/L, at least about 130 g/L, at least about 135 g/L, at least about 140 g/L, at least about 145 g/L, at least about 150 g/L, at least about 155 g/L, at least about 160 g/L, at least about 165 g/L, at least about 170 g/L, at least about 175 g/L, at least about 180 g/L, at least about 185 g/L, at least about 190 g/L, at least about 195 g/L or at least about 200 g/L.

In some embodiments, the recombinant protein is expressed in the algae chloroplast. For example, a recombinant gene of interest is driven using the 16S promoter of the endogenous chloroplast genome. In various embodiments, a recombinant gene of interest is driven using the 16S promoter from a Chlamydomonas species. In various embodiments, a recombinant gene of interest is driven using the 16S promoter set forth in SEQ ID NO: 1. In some instances, the promoter can be synthetically combined with a non-native untranslated region. Exemplary untranslated regions that can be employed include the 5′UTR of any of the following genes: psbE, psbI, psbK, rpL14, rpoB-2, atpF, clpP, petA, petB, petG, psaA, psaB, rps18, rps19, tufA, ycF4, rps14, or rps7. In some embodiments, an exogenous DNA construct encoding a recombinant protein of interest is recombined into the chloroplast genome of the algae, such as into the chloroplast of a Chlamydomonas species using any techniques available in the art.

### Growth Vessels

The microalgae useful for practicing the methods disclosed herein can be grown on land, for example, in ponds, aqueducts, or in closed or partially closed bioreactor systems. The algae can also be grown directly in water, for example, in an ocean, sea, lake, river, reservoir, etc. In various embodiments, the algae may be grown in culture systems of different volumes. In various embodiments, the algae can be grown, for example, in small scale laboratory systems. Small scale laboratory systems refer to cultures in volumes of less than about 6 liters. In various embodiments, the small scale laboratory culture may be about 1 liter, about 2 liters, about 3 liters, about 4 liters, or about 5 liters. In other embodiments, the small scale laboratory culture may be less than one liter. In yet other embodiments, the small scale laboratory culture may be 100 milliliters or less. In various embodiments, the culture may be 10 milliliters or less. In various embodiments, the culture may be 5 milliliters or less. In yet other embodiments, the culture may be 1 milliliter or less.

Alternatively, the culture systems may be large scale cultures, where large scale cultures refers to growth of cultures in volumes of greater than about 6 liters, greater than about 10 liters, or greater than about 20 liters. Large scale growth can also be growth of cultures in volumes of about 50 liters or more, about 100 liters or more, or about 200 liters or more. Large scale growth can be growth of cultures in, for example, ponds, containers, vessels, or other areas, where the pond, container, vessel, or area that contains the culture is for example, at least about 5 square meters, at least about 10 square meters, at least about 200 square meters, at least about 500 square meters, at least about 1,500 square meters, at least about 2,500 square meters, in area, or greater.

The present disclosure further provides for production of algae in very large scale culture systems. A very large scale liquid culture system may be from about 10,000 to about 20,000 liters. In various embodiments, the very large scale culture system may be from about 10,000 to about 40,000 liters or from about 10,000 to about 80,000 liters. In other embodiments, the very large scale culture system may be from about 10,000 to about 100,000 liters or from about 10,000 to about 150,000 liters. In yet other embodiments, the culture system may be from about 10,000 to about 200,000 liters or from about 10,000 to about 250,000 liters. The present disclosure also includes culture systems from about 10,000 to about 500,000 liters or from about 10,000 to about 600,000 liters. The present disclosure further provides for culture systems from about 10,000 to about 1,000,000 liters.

In various embodiments, the culture system may be a pond, either natural or artificial. In certain embodiments, the artificial pond may be a raceway pond. In a raceway pond, the algae, water, and nutrients circulate around a “racetrack.” Means of motivation, such as paddlewheels, provide constant motion to the liquid in the racetrack, allowing for the organism to be circulated back to the surface of the liquid at a chosen frequency. Paddlewheels also provide a source of agitation and oxygenate the system. CO₂ may be added to a culture system as a feedstock for photosynthesis through a CO₂ injection system. These raceway ponds can be enclosed, for example, in a building or a greenhouse, or can be located outdoors. In various embodiments, an outdoor raceway culture system may be enclosed with a cover or may be exposed to the environment.

Alternatively, microalgae can be grown in closed structures such as bioreactors, where the environment is under stricter control than in open systems or semi-closed systems. A photobioreactor is a bioreactor which incorporates some type of light source to provide photonic energy input into the reactor. The term “bioreactor” can refer to a system closed to the environment and having no direct exchange of gases and contaminants with the environment. Thus, a bioreactor can be described as an enclosed, and in the case of a photobioreactor, illuminated, culture vessel designed for controlled biomass production of liquid cell suspension cultures. Examples of bioreactors include, but are not limited to, glass containers, stainless steel containers, plastic tubes, tanks, plastic sleeves, and bags. In the case of photobioreactors, examples of light sources that can be used include, but not limited to, fluorescent bulbs, LEDs, and natural sunlight. Because these systems are closed, everything that the organism needs to grow (for example, carbon dioxide, nutrients, water, and light) must be introduced into the bioreactor.

Bioreactors, despite the costs to set up and maintain, have several advantages over open systems. They can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations. In some embodiments, the methods described herein are performed in a high-density fermenter.

### Harvesting

Microalgae can be continually harvested (as is with the majority of the larger volume cultivation systems), or harvested one batch at a time (for example, as with polyethylene bag cultivation). Batch harvesting is set up with, for example, nutrients, an organism (for example, microalgae), and water, where the organism is allowed to grow until the batch is harvested. With continuous harvesting, a portion of the algal mass can be harvested, for example, either continually, daily, or at fixed time intervals.

Harvesting of algae cultures may be accomplished by any method known in the art, including, but not limited to, filtration, batch centrifugation or continuous centrifugation. In some embodiments, the production culture reaches the harvest density within about 96 hours after the start of the culture. In some embodiments, the production culture reaches the harvest density within about % hours, about 120 hours, about 150 hours, about 175 hours, about 200 hours, about 220 hours, or about 250 hours after the start of the culture. In some embodiments, the production culture reaches the harvest density within about 250 hours of the start of the culture.

In some embodiments, the algae is dried after harvesting by, for example, spray drying, ring drying, paddle drying, tray drying, solar or sun drying, vacuum drying or freeze drying. Thus, in various embodiments, the harvested algae may be dried, for example to a moisture content of not more than about 15%. In still other embodiments the method further comprises isolating the at least one therapeutic protein from the algae.

Harvesting as it relates to production of a protein, including one or more heterologous proteins can be accomplished by any methods known in the art. For example, protein may be harvested as a whole biomass from the algal culture, as a fractionated biomass or from the media external to the algal cells. Protein can be further purified if desired by biochemical, physical and affinity means known in the art.

### Example 1

### Molecular Constructs for Expression of Recombinant Proteins in Dark or Limited Light Conditions

A library of expression cassettes was designed and constructed. Each cassette had a 5′-untranslated region from one of psaA (SEQ ID NO: 12), psaB (SEQ ID NO: 13), clpP (SEQ ID NO: 14), psbI (SEQ ID NO: 15), psbK (SEQ ID NO: 16), psbM (SEQ ID NO: 17), rpl4 (SEQ ID NO: 18), rps7 (SEQ ID NO: 19), rps14 (SEQ ID NO: 20), or rps19 (SEQ ID NO: 21) genes. The sequence of each 5′-untranslated region was amplified from the C. reinhardtii chloroplast genome. Each amplified 5′-untranslated region was separately ligated downstream of the 16S promoter (SEQ ID NO: 1). The 5′-untranslated regions were positioned in each expression cassette upstream to the insertion site for a gene of interest. Each of these expression cassettes allows a gene of interest to be expressed in the dark or light limited conditions.

16S Promoter (SEQ ID NO: 1):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagt
16S Promoter-psaA 5′UTR (SEQ ID NO: 2):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagtcttttacgaatacacatatggtaaaaaat
aaaacaatatctttaaaataagtaaaaataatttgtaaaccaataaaaaatatatttatggt
ataatataacatatgatgtaaaaaaaactatttgtctaatttaataaccatgcattttttat
gaacacataataattaaaagcgttgctaatggtgtaaataatgtatttattaaattaaataa
ttgttattataaggagaaatcc
16S Promoter-psaB 5′UTR (SEQ ID NO: 3):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttttgaattaaaatttcccacaggattatg
gcgtagtcataatatcaactaaaaaatctttttaaattttaaaatttacttttttacgcttt
tgtatgcaaagtttgctttgcacctgaatagttttattaaatttttatttaatggtagttta
atagtagtaatttacttcaattaaacaaaaaaaatcctaattgtttatccctttaaaagagc
gcttaaagtttttttacttagtgaagtaaaaataccgctcccttctggtattttttcttttg
atttaacaattagcattttaaccttttacttttctctcagtgttatactgcttaaaagtttt
taggtcattagataatatttaataatattacatatagggagtaagacaatttt
16S Promoter-clpP 5′UTR (SEQ ID NO: 4):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagtagttatattctggttaaaggatcggaact
aaccccaagtctctagtctaaacaaaaaattgtgtatgcatttaacacatttagtgttttta
actagacaaaaaaaattaagtatgatattataaaagtaatattttttagccttcgtgatgga
actggtagacatcctggttttaggaaccagtgctgaaaggcgtgccggttcaaatccggccg
aaggcattttaagtttaacgtagagccaatatttgtttgaatttatctattttttaaaccat
tttggtttaaaatttttatttgcttcaaaggagcctgtaaacggtactttaatttttacagt
agcactcgcagagcttatttacgtgcaaataaaagctctatctactaggatattagactagt
attaataaaacacaacattttattaacaaagtaattt
16S Promoter-psbI 5′UTR (SEQ ID NO: 5):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttgctcttttggggtcttattagctagtat
tagttaactaacaaaagatcaatattttagtttgttttatatattttattacttaagtagta
aggatttgcatttagcaatcttaaatacttaagtaataatctataaataaaatatattttcg
ctttaaaacttataaaaattatttgctcgttataagcctaaaaaaacgtaggatctctacga
gatattacattgtttttttctttaattggctttaatattactttgtatatataaaccaaagt
acttgttaatagttattaaattatattaactatacagtacaaagaaattttttgctaaaaaa
agt
16S Promoter-psbK 5′UTR (SEQ ID NO: 6):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttggcgttcgatttcttgaacacttaagag
aatttttattttagaaagaaaaaacgagctttaaggtgagcttattttgttgcgtgtaaatt
tttaaaatctaaggtgtatagacaaaaatctacattttcatatgctaaaaacatactcttta
cgggtacgcgaatgttaggtaaattttcacaactaactctatggttgtgggaagaaaaccaa
atacatagagatatttttaaaaagatatctctcactttaatagattttattataaatactat
caacaatttcttaaactttttaagaaggatattt
16S Promoter-psbM 5′UTR (SEQ ID NO: 7):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttaccgaatttgctggcatctaaaaaattt
taacctttagatctctgcatagagtatttcctacaaagtacttaatttattacaatatattt
ttaacctaaaaggtaaaccttaagaacgtagttggatcattgtcagaatcttgcacttttgg
gtcaataaaatatttattgacccactttgctccctaaactattggagatgcaactaccatta
aaatacgtctccactttgtaactctagacggtatgtcaatattcttgatcaaaagggagtta
ctaacaaagaaattttaagtttaaaatttttataaaaagttttattaatataact
16S Promoter-rpL14 5′UTR (SEQ ID NO: 8):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagtatttaaaaaatatttaagaaaattaagag
cataagtattgtttcgctttggctcaaaagccaatactaaagataatattactttttgtaag
tttttacttactcggtttgtaccaggcaaccctataaatatagtaaaatggaattaaactag
atatatctctttaagaaagattttctcatcaaggctgccctttaactttaacctagaatgac
taaaaggagtaagcaaataccgagaaatttattttttcacttaatgaaaaaataaattttat
ctctttctcttttaagcatataaatatgaaggtaagtaaactctactagggaaaagcatagt
gttgaaggatatactttcttgggatccaaaaaagtaaacctaaacaagatatacttaattaa
tgataataatataaaacttttttttaaactt
16S Promoter-rps7 5′UTR (SEQ ID NO: 9):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttgcatatctattaagtagcgattttcaaa
gaggcagttggcaggacgtccccttacgggaatataaatattagtggcagtggtaccgccac
tgcctatatttatatactccgaaggaacttgttagccgataggcgaggcaacaaatttattt
attgtatataaatatccactaaaatttatttgcccgaaggggacgtcctattaaaccatcac
ataactaaaattgcttatttggtatgaaagtttgcatctattttaaccatttagtaaaaata
atgatgcttttttaaaataaaa
16S Promoter-rps14 5′UTR (SEQ ID NO: 10):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagtgacaactaacagtctttattcctaatttt
acttcggagcaacgaaattgtctttctctccgttagagaaaacaaattgcgaagcatccatt
tacccattagagaaagactaaagtttatctctagagtggtatgcctctaggtaaaggacgtt
ttaaaagggtaatttattaaatatagataaatcgtgtcagtttttgaattgatagctttttt
ataacagtaaaataataattgttttcttttatatttattactgattttcgatttctgctggg
caacattctccttccgagtagggacatgtaccaagtcatccttcttttatttgaataataaa
aataaataatataaaatggaatttaaaat
16S Promoter-rps19 5′UTR (SEQ ID NO: 11):
ggcaggcaacaaatttatttattgtcccgtaaggggaaggggaaaacaattattattttact
gcggagcagcttgttattagaaatttttattaaaaaaaaaataaaaatttgacaaaaaaaaa
taaaaaagttaaattaaaaacactgggaatgttctaacaatcataaaaaaatcaaaagggtt
taaaatcccgacaaaatttaaactttaaagagttaaatatcggcagttggcaggcaactgcc
actgacgtccactaaaatttattctttctcggggacaataaataaatttgtcctgtaaaggg
acgtaaaatagcagtaagcataagtatggccacttgcttaaattttacaatattaaaaaaat
tctagaaataataaagttttggttgataaatttttaacgttaattgtttgtttaaactttat
agatatcgggacttagtaagtctaaagtcgctaaaaacaaccagtttcagataaacatttgt
ttcaactgattggttcgttttgtttatccttagagtttatatatcttaactctatattgggt
aaaccactataatggtcatatgttggaaaaattccaataaatttcaatttaatgtggaattt
aaaaagctcatatgtacttaaaatagacaattgttaaacatgaatagaaaatattacctact
tttatttttataaatacagctttagccattattataaaattcaaaagtcattttaaaaaatc
aa
psaA 5′UTR (SEQ ID NO: 12):
cttttacgaatacacatatggtaaaaaataaaacaatatctttaaaataagtaaaaataatt
tgtaaaccaataaaaaatatatttatggtataatataacatatgatgtaaaaaaaactattt
gtctaatttaataaccatgcattttttatgaacacataataattaaaagcgttgctaatggt
gtaaataatgtatttattaaattaaataattgttattataaggagaaatcc
psaB 5′UTR (SEQ ID NO: 13):
tttgaattaaaatttcccacaggattatggcgtagtcataatatcaactaaaaaatcttttt
aaattttaaaatttacttttttacgcttttgtatgcaaagtttgctttgcacctgaatagtt
ttattaaatttttatttaatggtagtttaatagtagtaatttacttcaattaaacaaaaaaa
atcctaattgtttatccctttaaaagagcgcttaaagtttttttacttagtgaagtaaaaat
accgctcccttctggtattttttcttttgatttaacaattagcattttaaccttttactttt
ctctcagtgttatactgcttaaaagtttttaggtcattagataatatttaataatattacat
atagggagtaagacaatttt
clpP 5′UTR (SEQ ID NO: 14):
agttatattctggttaaaggatcggaactaaccccaagtctctagtctaaacaaaaaattgt
gtatgcatttaacacatttagtgtttttaactagacaaaaaaaattaagtatgatattataa
aagtaatattttttagccttcgtgatggaactggtagacatcctggttttaggaaccagtgc
tgaaaggcgtgccggttcaaatccggccgaaggcattttaagtttaacgtagagccaatatt
tgtttgaatttatctattttttaaaccattttggtttaaaatttttatttgcttcaaaggag
cctgtaaacggtactttaatttttacagtagcactcgcagagcttatttacgtgcaaataaa
agctctatctactaggatattagactagtattaataaaacacaacattttattaacaaagta
attt
psbI 5′UTR (SEQ ID NO: 15):
gtgctcttttggggtcttattagctagtattagttaactaacaaaagatcaatattttagtt
tgttttatatattttattacttaagtagtaaggatttgcatttagcaatcttaaatacttaa
gtaataatctataaataaaatatattttcgctttaaaacttataaaaattatttgctcgtta
taagcctaaaaaaacgtaggatctctacgagatattacattgtttttttctttaattggctt
taatattactttgtatatataaaccaaagtacttgttaatagttattaaattatattaacta
tacagtacaaagaaattttttgctaaaaaaagt
psbK 5′UTR (SEQ ID NO: 16):
tggcgttcgatttcttgaacacttaagagaatttttattttagaaagaaaaaacgagcttta
aggtgagcttattttgttgcgtgtaaatttttaaaatctaaggtgtatagacaaaaatctac
attttcatatgctaaaaacatactctttacgggtacgcgaatgttaggtaaattttcacaac
taactctatggttgtgggaagaaaaccaaatacatagagatatttttaaaaagatatctctc
actttaatagattttattataaatactatcaacaatttcttaaactttttaagaaggatatt
t
psbM 5′UTR (SEQ ID NO: 17):
taccgaatttgctggcatctaaaaaattttaacctttagatctctgcatagagtatttccta
caaagtacttaatttattacaatatatttttaacctaaaaggtaaaccttaagaacgtagtt
ggatcattgtcagaatcttgcacttttgggtcaataaaatatttattgacccactttgctcc
ctaaactattggagatgcaactaccattaaaatacgtctccactttgtaactctagacggta
tgtcaatattcttgatcaaaagggagttactaacaaagaaattttaagtttaaaatttttat
aaaaagttttattaatataact
rpL14 5′UTR (SEQ ID NO: 18):
atttaaaaaatatttaagaaaattaagagcataagtattgtttcgctttggctcaaaagcca
atactaaagataatattactttttgtaagtttttacttactcggtttgtaccaggcaaccct
ataaatatagtaaaatggaattaaactagatatatctctttaagaaagattttctcatcaag
gctgccctttaactttaacctagaatgactaaaaggagtaagcaaataccgagaaatttatt
ttttcacttaatgaaaaaataaattttatctctttctcttttaagcatataaatatgaaggt
aagtaaactctactagggaaaagcatagtgttgaaggatatactttcttgggatccaaaaaa
gtaaacctaaacaagatatacttaattaatgataataatataaaacttttttttaaactt
rps7 5′UTR (SEQ ID NO: 19):
tgcatatctattaagtagcgattttcaaagaggcagttggcaggacgtccccttacgggaat
ataaatattagtggcagtggtaccgccactgcctatatttatatactccgaaggaacttgtt
agccgataggcgaggcaacaaatttatttattgtatataaatatccactaaaatttatttgc
ccgaaggggacgtcctattaaaccatcacataactaaaattgcttatttggtatgaaagttt
gcatctattttaaccatttagtaaaaataatgatgcttttttaaaataaaa
rps14 5′UTR (SEQ ID NO: 20).
gacaactaacagtctttattcctaattttacttcggagcaacgaaattgtctttctctccgt
tagagaaaacaaattgcgaagcatccatttacccattagagaaagactaaagtttatctcta
gagtggtatgcctctaggtaaaggacgttttaaaagggtaatttattaaatatagataaatc
gtgtcagtttttgaattgatagcttttttataacagtaaaataataattgttttcttttata
tttattactgattttcgatttctgctgggcaacattctccttccgagtagggacatgtacca
agtcatccttcttttatttgaataataaaaataaataatataaaatggaatttaaaat
rps19 5′UTR (SEQ ID NO: 21):
taaatatcggcagttggcaggcaactgccactgacgtccactaaaatttattctttctcggg
gacaataaataaatttgtcctgtaaagggacgtaaaatagcagtaagcataagtatggccac
ttgcttaaattttacaatattaaaaaaattctagaaataataaagttttggttgataaattt
ttaacgttaattgtttgtttaaactttatagatatcgggacttagtaagtctaaagtcgcta
aaaacaaccagtttcagataaacatttgtttcaactgattggttcgttttgtttatccttag
agtttatatatcttaactctatattgggtaaaccactataatggtcatatgttggaaaaatt
ccaataaatttcaatttaatgtggaatttaaaaagctcatatgtacttaaaatagacaattg
ttaaacatgaatagaaaatattacctacttttatttttataaatacagctttagccattatt
ataaaattcaaaagtcattttaaaaaatcaa

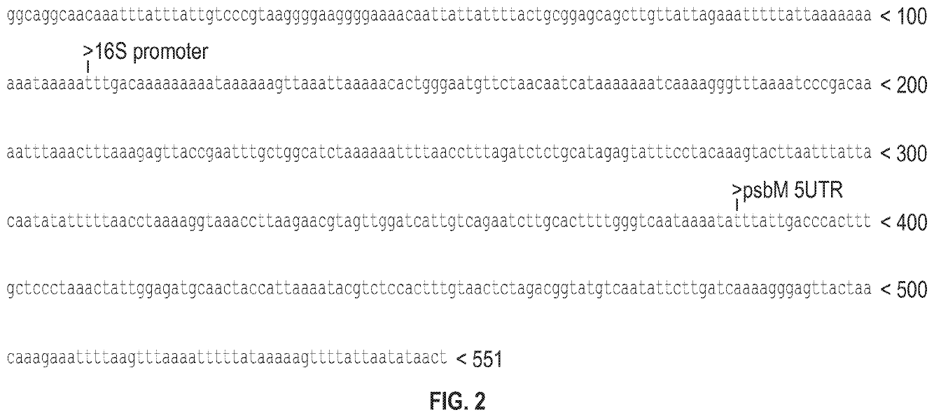

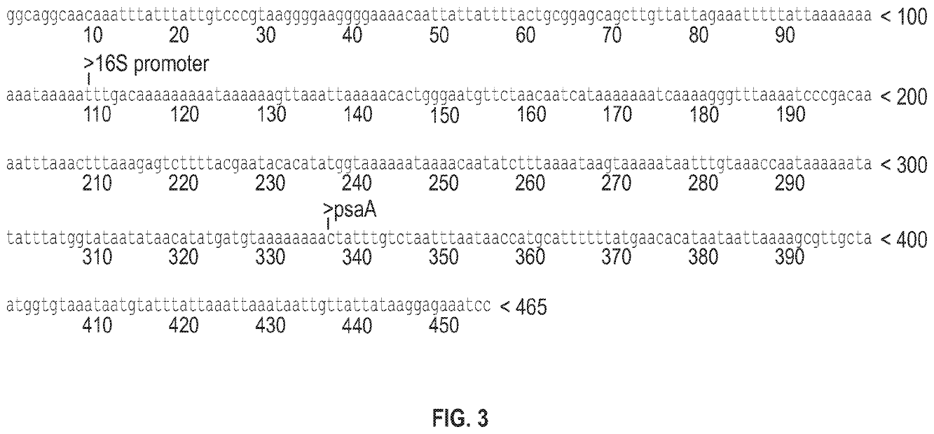

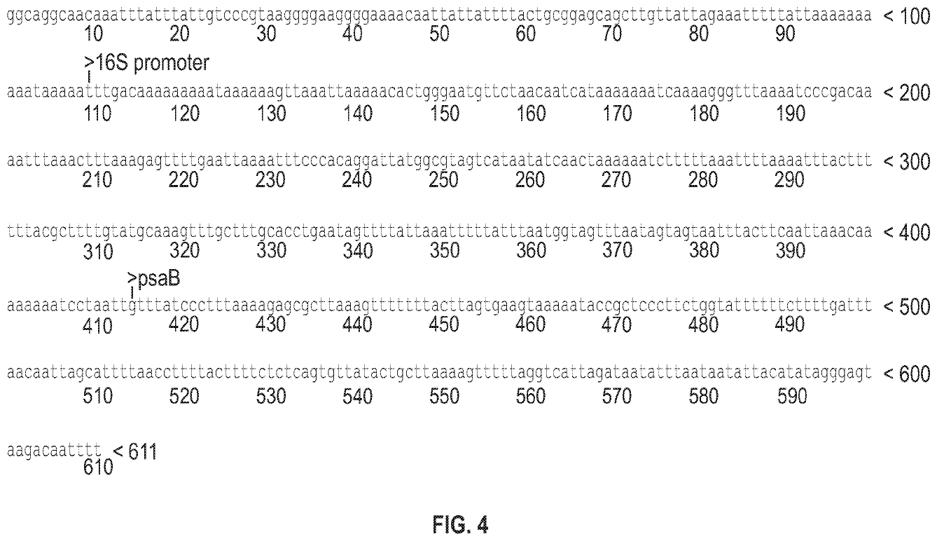

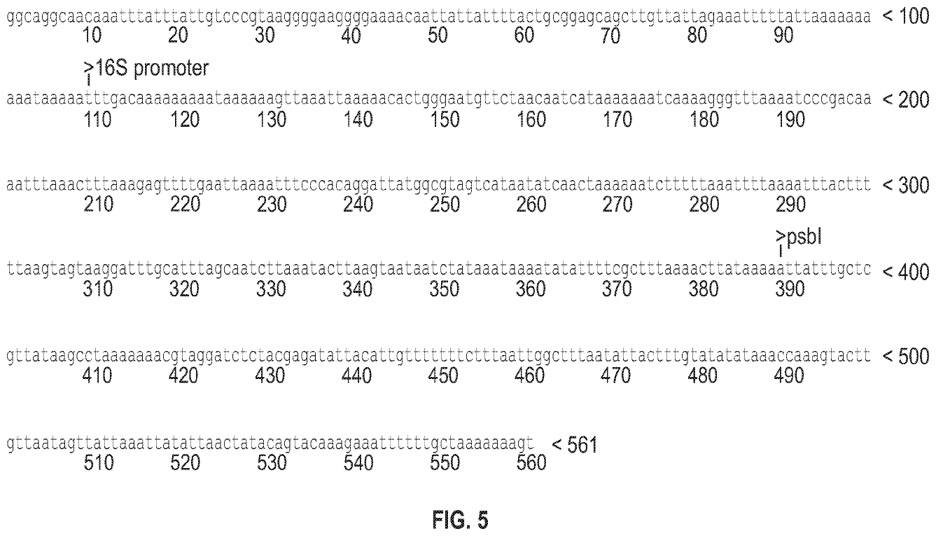

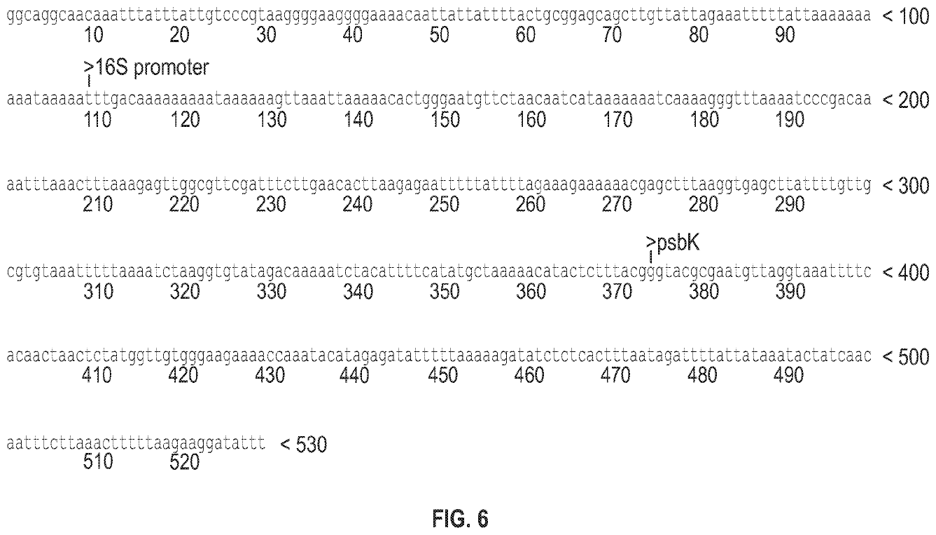

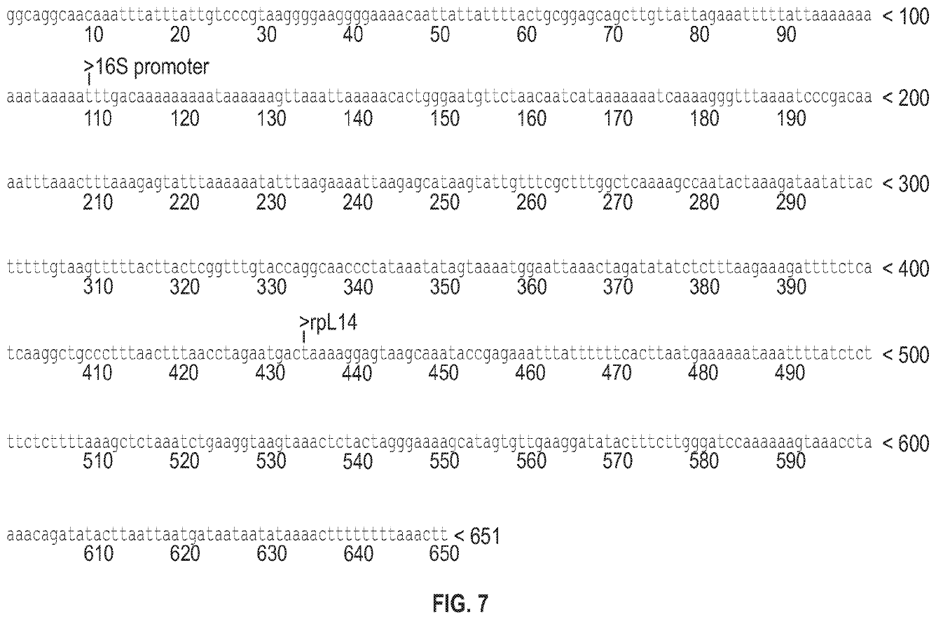

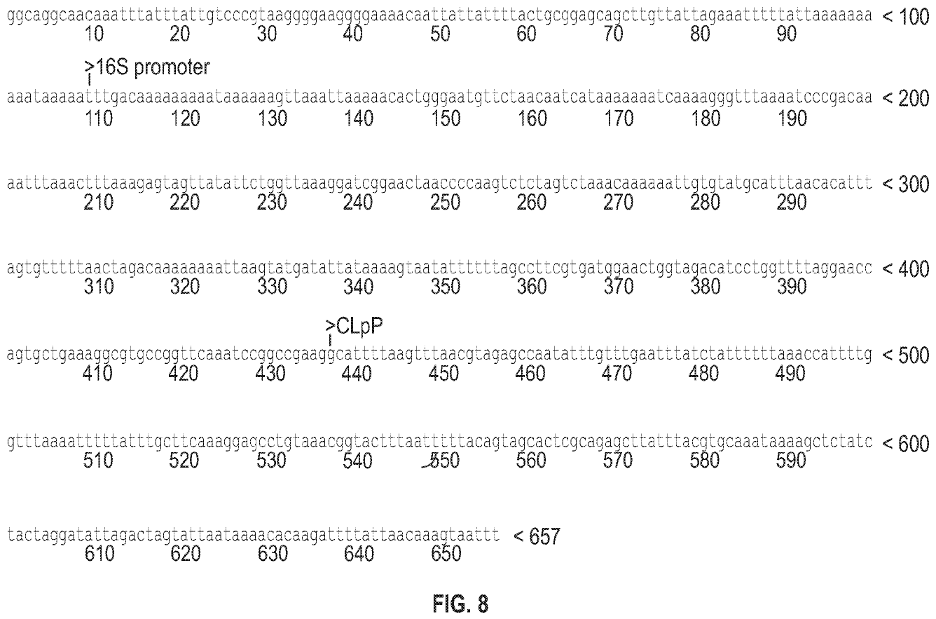

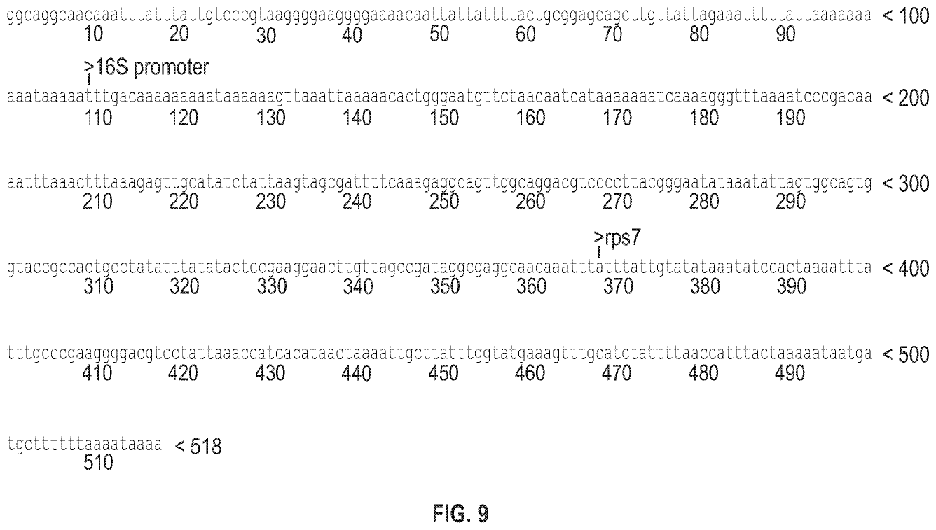

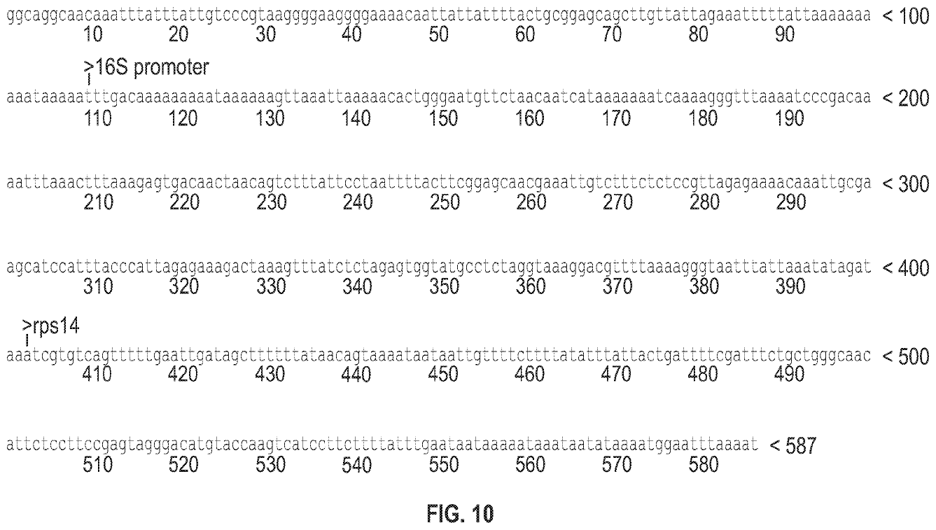

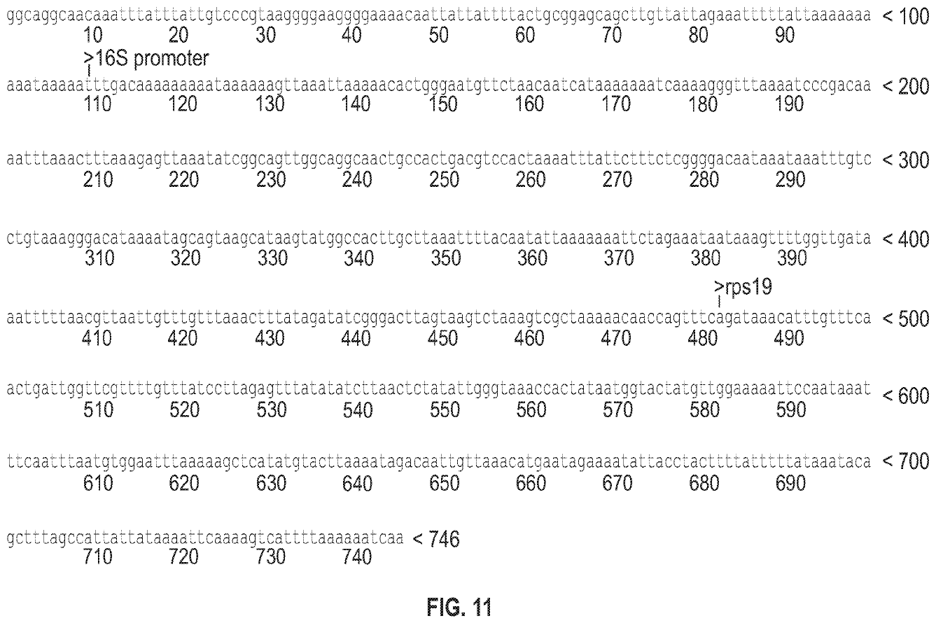

The expression cassettes were further designed and constructed to include additional elements. A gene of interest encoding the recombinant protein (RP) was cloned upstream of a 3′-untranslated region of the rbcL chloroplast gene. The vectors also contained a selection cassette that includes the psbD promoter and 5′-UTR, the apaVI kanamycin resistance gene, and a second rbcL 3′-UTR. The selection cassette allows algae transformed with the DNA construct to survive on medium that contains the antibiotic kanamycin. The DNA construct also contains homology on the 5′ and 3′ regions that allow the constructs to integrate into a silent site of the C. reinhardtii chloroplast genome upstream of the psbH and psbN genes. The constructs described above are shown in FIGS. 2-11.

### Example 2

### Recombinant Protein Accumulation

The gene of interest for the excombinant protein (RP) was placed under the control of a variety of promoters in the following expression constructs: psbA promoter and UTR (positive control)

16S promoter and psaA 5′-UTR (SEQ ID NO: 2),

16S promoter and psaB 5′-UTR (SEQ ID NO: 3),

16S promoter and clpP 5′UTR (SEQ ID NO: 4),

16S-promoter and psbI 5′ UTR (SEQ ID NO: 5),

16S-promoter and psbK 5′-UTR (SEQ ID NO: 6),

16S-promoter and psbM 5′-UTR (SEQ ID NO: 7),

16S-promoter and rpl4 5′-UTR (SEQ ID NO: 8),

16S-promoter and rps7 5′-UTR (SEQ ID NO: 9),

16S-promoter and rps14 5′-UTR (SEQ ID NO: 10), and

16S-promoter and rps19-UTR (SEQ ID NO: 11).

The negative control was a wild-type control strain grown in the light and the positive control was a strain of algae transformed with the psbA promoter and UTR driving the expression of RP. Each of the constructs included a FLAG tag fused at the N-terminus of the protein coding region. Each of the constructs with a 16S-promoter was transformed into C. reinhardtii.

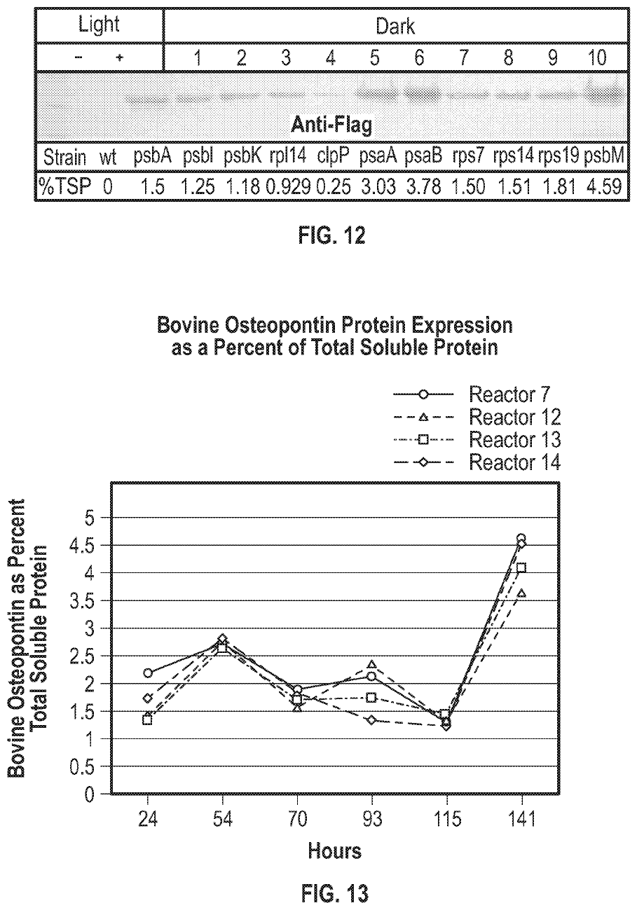

Each transformed strain was then grown in the dark to determine their ability to allow for protein accumulation in dark or light limited conditions. Once grown to a stationary phase, cells were spun down by centrifugation. Cells were lysed by sonication in Tris-buffered saline pH 8.0. Twenty (20) μg of each soluble protein lysate was then separated by gel electrophoresis and transferred to a nitrocellulose membrane. The nitrocellulose membrane was then probed with an anti-FLAG antibody to determine the ability of each expression construct to provide for protein expression and accumulation in the dark. Enzyme-linked immunoabsorbant assay was used to determine the percent RP as a proportion of total soluble protein (TSP). The results are shown in FIG. 12. The RP used in this example is bovine osteopontin.

### Example 3

### Protein Accumulation in Dark Conditions

Cells transformed with the expression construct that included the 16S-promoter and psbM 5′-UTR upstream of bovine osteopontin were grown in dark fermentation conditions. Acetate was used as a substrate to allow for heterotrophic grown in the dark. Accumulation of recombinant bovine osteopontin was monitored by enzyme-linked immunoabsorbant assay at various time intervals. The results of protein accumulation in 4 fermenter runs are shown in FIG. 13.

### Example 4

### Growth on Exogenous Organic Carbon Sources

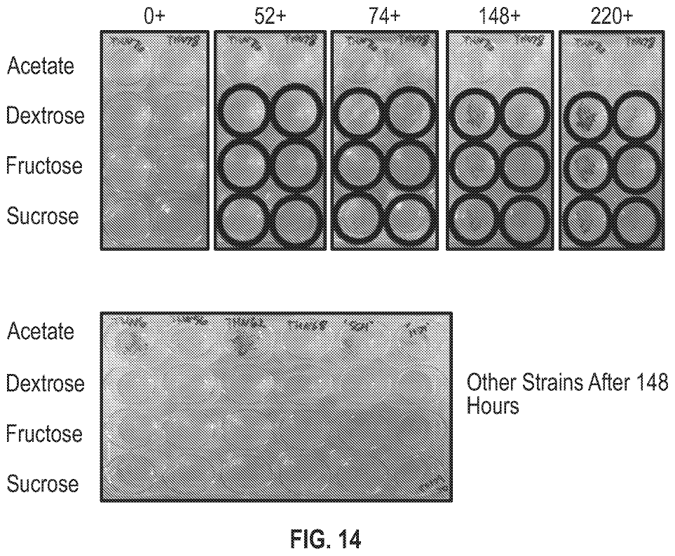

Strains of C. reinhardtii algae were struck out on media containing the exogenous carbon sources acetate, dextrose, fructose and glucose. The strains were observed at time points from 0 to about 220 hours for growth under dark conditions. Two strains, THN76 and THN78, demonstrated the ability to grow on dextrose, fructose, and sucrose. These two strains also exhibited some growth on acetate, albeit at a slower growth rate. In comparison, after growth for 148 hours, the remainder of the strains tested, THN6, THN56, THN62, THN68, 564 and 1171, did not exhibit growth on dextrose, fructose and glucose. Strains THN6, THN62 and 564 grew on acetate as the carbon source. Strains were reconfirmed as Chlamydomonas by ITS1 and ITS2 genetic sequencing. Growth results are shown in FIG. 14.

Although the invention has been described with reference to the above, it will be understood that modifications and variations are encompassed within the spirit and scope of the invention. Accordingly, the invention is limited only by the following claims.

1. A method for producing a high-density culture of an algae species comprising:
growing an algae species in the presence of at least one exogenous organic carbon source under aerobic conditions, wherein the algae species is capable of using the organic carbon source as an energy source for growth, and wherein the algae species lacks a chitin cell wall.

2. The method of claim 1, wherein the algae species is a Chlamydomonas species.

3. (canceled)

4. The method of claim 1, wherein one of the at least one exogenous carbon source is selected from the group consisting of glucose, fructose, sucrose, maltose, glycerol, molasses, starch, cellulose, acetate, and any combination thereof.

5. The method of claim 4, wherein the algae species is a Chlamydomonas species and the Chlamydomonas species is grown in the presence of light.

6. The method of claim 4, wherein the algae species is a Chlamydomonas species and the Chlamydomonas species is grown in limited light conditions or in the dark.

7. (canceled)

8. The method of claim 6, wherein the Chlamydomonas species is grown to a density of at least 30 g/L.

9. (canceled)

10. (canceled)

11. The method of claim 2, wherein the Chlamydomonas sp. is one or more of Chlamydomonas reinhardtii, Chlamydomonas dysomos, Chlamydomonas mundane, Chlamydomonas debaryana, Chlamydomonas moewusii, Chlamydomonas culleus, Chlamydomonas noctigama, Chlamydomonas aulata, Chlamydomonas applanata, Chlamydomonas marvanii, and Chlamydomonas proboscigera.

12. The method of claim 11, wherein the Chlamydomonas species is Chlamydomonas reinhardtii and wherein the organic carbon source is acetate.

13. A method for accumulating a recombinant protein from a culture of a Chlamydomonas species comprising:
(a) providing one or more cells of a recombinant Chlamydomonas species capable of expressing a recombinant protein, wherein the Chlamydomonas species lacks a chitin cell wall;
(b) growing the one or more cells in the presence of at least one exogenous organic carbon source under aerobic conditions to generate a culture of the recombinant Chlamydomonas species, wherein the Chlamydomonas species uses the organic carbon source as an energy source for growth; and
(c) harvesting the recombinant protein from the culture.

14. The method of claim 13, wherein one of the at least one exogenous carbon source is selected from the group consisting of glucose, fructose, sucrose, maltose, glycerol, molasses, starch, cellulose, acetate, and any combination thereof.

15-17. (canceled)

18. The method of claim 6, wherein exogenous air or oxygen is supplied during the growing step.

19-26. (canceled)

27. The method of claim 2, wherein productivity of Chlamydomonas cultivation in grams (g) of Chlamydomonas biomass per liter (L) of culture is at least about 0.3 g/L/hour.

28. The method of claim 2, wherein conversion efficiency of Chlamydomonas biomass on the exogenous organic carbon source is at least about 0.3 g biomass/g carbon source.

29. The method of claim 28, wherein total protein content of Chlamydomonas biomass of the Chlamydomonas culture is at least about 20%.

30. (canceled)

31. An expression cassette comprising an algae 16S promoter fused to a 5′-untranslated region (5′ UTR) and a nucleic acid molecule encoding a recombinant protein, wherein the 5′UTR is selected from the group consisting of psbM, psaA, psaB, psbI, psbK, clpP, rpl4, rps7, rps14, and rps19 5′UTR.

32-37. (canceled)

38. A method of expressing a recombinant protein in an algae comprising:
(a) introducing the expression cassette of claim 31 into an algae, and
(b) growing the algae under dark or limited light conditions,
wherein the 5′UTR is:
(i) selected from the group consisting of psbM, psaA, psaB, psbI, psbK, clpP, rpl14, rps7, rps14, and rps19 5′UTR;
(ii) a sequence selected from the group consisting of SEQ ID NOs:12-20 and 21; or
(iii) comprises a sequence with at least 80% sequence identity to a sequence selected from the group consisting of SEQ ID NOs: 12-20 and 21.

39-44. (canceled)

45. The method of claim 6, wherein productivity of Chlamydomonas cultivation in grams (g) of Chlamydomonas biomass per liter (L) of culture is at least about 0.3 g/L/hour.

46. The method of claim 6, wherein conversion efficiency of Chlamydomonas biomass on the exogenous organic carbon source is at least about 0.3 g biomass/g carbon source.

47. The method of claim 46, wherein total protein content of Chlamydomonas biomass of the Chlamydomonas culture is at least about 20%.


SEQUENCE LISTING (21 sequences)

SEQ ID NO: 1 - DNA, 219 bp, Chlamydomonas reinhardtii
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagt

SEQ ID NO: 2 - DNA, 456 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagtcttttacgaatacacatatggtaaaaaataaaacaatatctttaaaataagtaaaaataatttgtaaaccaataaaaaatatatttatggtataatataacatatgatgtaaaaaaaactatttgtctaatttaataaccatgcattttttatgaacacataataattaaaagcgttgctaatggtgtaaataatgtatttattaaattaaataattgttattataaggagaaatcc

SEQ ID NO: 3 - DNA, 611 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttttgaattaaaatttcccacaggattatggcgtagtcataatatcaactaaaaaatctttttaaattttaaaatttacttttttacgcttttgtatgcaaagtttgctttgcacctgaatagttttattaaatttttatttaatggtagtttaatagtagtaatttacttcaattaaacaaaaaaaatcctaattgtttatccctttaaaagagcgcttaaagtttttttacttagtgaagtaaaaataccgctcccttctggtattttttcttttgatttaacaattagcattttaaccttttacttttctctcagtgttatactgcttaaaagtttttaggtcattagataatatttaataatattacatatagggagtaagacaatttt

SEQ ID NO: 4 - DNA, 657 bp, Artificial Sequence
  Synthetic Construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagtagttatattctggttaaaggatcggaactaaccccaagtctctagtctaaacaaaaaattgtgtatgcatttaacacatttagtgtttttaactagacaaaaaaaattaagtatgatattataaaagtaatattttttagccttcgtgatggaactggtagacatcctggttttaggaaccagtgctgaaaggcgtgccggttcaaatccggccgaaggcattttaagtttaacgtagagccaatatttgtttgaatttatctattttttaaaccattttggtttaaaatttttatttgcttcaaaggagcctgtaaacggtactttaatttttacagtagcactcgcagagcttatttacgtgcaaataaaagctctatctactaggatattagactagtattaataaaacacaacattttattaacaaagtaattt

SEQ ID NO: 5 - DNA, 561 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagt

SEQ ID NO: 6 - DNA, 530 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttggcgttcgatttcttgaacacttaagagaatttttattttagaaagaaaaaacgagctttaaggtgagcttattttgttgcgtgtaaatttttaaaatctaaggtgtatagacaaaaatctacattttcatatgctaaaaacatactctttacgggtacgcgaatgttaggtaaattttcacaactaactctatggttgtgggaagaaaaccaaatacatagagatatttttaaaaagatatctctcactttaatagattttattataaatactatcaacaatttcttaaactttttaagaaggatattt

SEQ ID NO: 7 - DNA, 551 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttaccgaatttgctggcatctaaaaaattttaacctttagatctctgcatagagtatttcctacaaagtacttaatttattacaatatatttttaacctaaaaggtaaaccttaagaacgtagttggatcattgtcagaatcttgcacttttgggtcaataaaatatttattgacccactttgctccctaaactattggagatgcaactaccattaaaatacgtctccactttgtaactctagacggtatgtcaatattcttgatcaaaagggagttactaacaaagaaattttaagtttaaaatttttataaaaagttttattaatataact

SEQ ID NO: 8 - DNA, 651 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagtatttaaaaaatatttaagaaaattaagagcataagtattgtttcgctttggctcaaaagccaatactaaagataatattactttttgtaagtttttacttactcggtttgtaccaggcaaccctataaatatagtaaaatggaattaaactagatatatctctttaagaaagattttctcatcaaggctgccctttaactttaacctagaatgactaaaaggagtaagcaaataccgagaaatttattttttcacttaatgaaaaaataaattttatctctttctcttttaagcatataaatatgaaggtaagtaaactctactagggaaaagcatagtgttgaaggatatactttcttgggatccaaaaaagtaaacctaaacaagatatacttaattaatgataataatataaaacttttttttaaactt

SEQ ID NO: 9 - DNA, 518 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttgcatatctattaagtagcgattttcaaagaggcagttggcaggacgtccccttacgggaatataaatattagtggcagtggtaccgccactgcctatatttatatactccgaaggaacttgttagccgataggcgaggcaacaaatttatttattgtatataaatatccactaaaatttatttgcccgaaggggacgtcctattaaaccatcacataactaaaattgcttatttggtatgaaagtttgcatctattttaaccatttagtaaaaataatgatgcttttttaaaataaaa

SEQ ID NO: 10 - DNA, 587 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagtgacaactaacagtctttattcctaattttacttcggagcaacgaaattgtctttctctccgttagagaaaacaaattgcgaagcatccatttacccattagagaaagactaaagtttatctctagagtggtatgcctctaggtaaaggacgttttaaaagggtaatttattaaatatagataaatcgtgtcagtttttgaattgatagcttttttataacagtaaaataataattgttttcttttatatttattactgattttcgatttctgctgggcaacattctccttccgagtagggacatgtaccaagtcatccttcttttatttgaataataaaaataaataatataaaatggaatttaaaat

SEQ ID NO: 11 - DNA, 746 bp, Artificial Sequence
  Synthetic construct
ggcaggcaacaaatttatttattgtcccgtaaggggaaggggaaaacaattattattttactgcggagcagcttgttattagaaatttttattaaaaaaaaaataaaaatttgacaaaaaaaaataaaaaagttaaattaaaaacactgggaatgttctaacaatcataaaaaaatcaaaagggtttaaaatcccgacaaaatttaaactttaaagagttaaatatcggcagttggcaggcaactgccactgacgtccactaaaatttattctttctcggggacaataaataaatttgtcctgtaaagggacgtaaaatagcagtaagcataagtatggccacttgcttaaattttacaatattaaaaaaattctagaaataataaagttttggttgataaatttttaacgttaattgtttgtttaaactttatagatatcgggacttagtaagtctaaagtcgctaaaaacaaccagtttcagataaacatttgtttcaactgattggttcgttttgtttatccttagagtttatatatcttaactctatattgggtaaaccactataatggtcatatgttggaaaaattccaataaatttcaatttaatgtggaatttaaaaagctcatatgtacttaaaatagacaattgttaaacatgaatagaaaatattacctacttttatttttataaatacagctttagccattattataaaattcaaaagtcattttaaaaaatcaa

SEQ ID NO: 12 - DNA, 237 bp, Artificial Sequence
  Synthetic construct
cttttacgaatacacatatggtaaaaaataaaacaatatctttaaaataagtaaaaataatttgtaaaccaataaaaaatatatttatggtataatataacatatgatgtaaaaaaaactatttgtctaatttaataaccatgcattttttatgaacacataataattaaaagcgttgctaatggtgtaaataatgtatttattaaattaaataattgttattataaggagaaatcc

SEQ ID NO: 13 - DNA, 392 bp, Artificial Sequence
  Synthetic construct
tttgaattaaaatttcccacaggattatggcgtagtcataatatcaactaaaaaatctttttaaattttaaaatttacttttttacgcttttgtatgcaaagtttgctttgcacctgaatagttttattaaatttttatttaatggtagtttaatagtagtaatttacttcaattaaacaaaaaaaatcctaattgtttatccctttaaaagagcgcttaaagtttttttacttagtgaagtaaaaataccgctcccttctggtattttttcttttgatttaacaattagcattttaaccttttacttttctctcagtgttatactgcttaaaagtttttaggtcattagataatatttaataatattacatatagggagtaagacaatttt

SEQ ID NO: 14 - DNA, 438 bp, Artificial Sequence
  Synthetic construct
agttatattctggttaaaggatcggaactaaccccaagtctctagtctaaacaaaaaattgtgtatgcatttaacacatttagtgtttttaactagacaaaaaaaattaagtatgatattataaaagtaatattttttagccttcgtgatggaactggtagacatcctggttttaggaaccagtgctgaaaggcgtgccggttcaaatccggccgaaggcattttaagtttaacgtagagccaatatttgtttgaatttatctattttttaaaccattttggtttaaaatttttatttgcttcaaaggagcctgtaaacggtactttaatttttacagtagcactcgcagagcttatttacgtgcaaataaaagctctatctactaggatattagactagtattaataaaacacaacattttattaacaaagtaattt

SEQ ID NO: 15 - DNA, 343 bp, Artificial Sequence
  Synthetic construct
gtgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagt

SEQ ID NO: 16 - DNA, 311 bp, Artificial Sequence
  Synthetic construct
tggcgttcgatttcttgaacacttaagagaatttttattttagaaagaaaaaacgagctttaaggtgagcttattttgttgcgtgtaaatttttaaaatctaaggtgtatagacaaaaatctacattttcatatgctaaaaacatactctttacgggtacgcgaatgttaggtaaattttcacaactaactctatggttgtgggaagaaaaccaaatacatagagatatttttaaaaagatatctctcactttaatagattttattataaatactatcaacaatttcttaaactttttaagaaggatattt

SEQ ID NO: 17 - DNA, 332 bp, Artificial Sequence
  Synthetic construct
taccgaatttgctggcatctaaaaaattttaacctttagatctctgcatagagtatttcctacaaagtacttaatttattacaatatatttttaacctaaaaggtaaaccttaagaacgtagttggatcattgtcagaatcttgcacttttgggtcaataaaatatttattgacccactttgctccctaaactattggagatgcaactaccattaaaatacgtctccactttgtaactctagacggtatgtcaatattcttgatcaaaagggagttactaacaaagaaattttaagtttaaaatttttataaaaagttttattaatataact

SEQ ID NO: 18 - DNA, 432 bp, Artificial Sequence
  Synthetic construct
atttaaaaaatatttaagaaaattaagagcataagtattgtttcgctttggctcaaaagccaatactaaagataatattactttttgtaagtttttacttactcggtttgtaccaggcaaccctataaatatagtaaaatggaattaaactagatatatctctttaagaaagattttctcatcaaggctgccctttaactttaacctagaatgactaaaaggagtaagcaaataccgagaaatttattttttcacttaatgaaaaaataaattttatctctttctcttttaagcatataaatatgaaggtaagtaaactctactagggaaaagcatagtgttgaaggatatactttcttgggatccaaaaaagtaaacctaaacaagatatacttaattaatgataataatataaaacttttttttaaactt

SEQ ID NO: 19 - DNA, 299 bp, Artificial Sequence
  Synthetic construct
tgcatatctattaagtagcgattttcaaagaggcagttggcaggacgtccccttacgggaatataaatattagtggcagtggtaccgccactgcctatatttatatactccgaaggaacttgttagccgataggcgaggcaacaaatttatttattgtatataaatatccactaaaatttatttgcccgaaggggacgtcctattaaaccatcacataactaaaattgcttatttggtatgaaagtttgcatctattttaaccatttagtaaaaataatgatgcttttttaaaataaaa

SEQ ID NO: 20 - DNA, 368 bp, Artificial Sequence
  Synthetic construct
gacaactaacagtctttattcctaattttacttcggagcaacgaaattgtctttctctccgttagagaaaacaaattgcgaagcatccatttacccattagagaaagactaaagtttatctctagagtggtatgcctctaggtaaaggacgttttaaaagggtaatttattaaatatagataaatcgtgtcagtttttgaattgatagcttttttataacagtaaaataataattgttttcttttatatttattactgattttcgatttctgctgggcaacattctccttccgagtagggacatgtaccaagtcatccttcttttatttgaataataaaaataaataatataaaatggaatttaaaat

SEQ ID NO: 21 - DNA, 527 bp, Artificial Sequence
  Synthetic construct
taaatatcggcagttggcaggcaactgccactgacgtccactaaaatttattctttctcggggacaataaataaatttgtcctgtaaagggacgtaaaatagcagtaagcataagtatggccacttgcttaaattttacaatattaaaaaaattctagaaataataaagttttggttgataaatttttaacgttaattgtttgtttaaactttatagatatcgggacttagtaagtctaaagtcgctaaaaacaaccagtttcagataaacatttgtttcaactgattggttcgttttgtttatccttagagtttatatatcttaactctatattgggtaaaccactataatggtcatatgttggaaaaattccaataaatttcaatttaatgtggaatttaaaaagctcatatgtacttaaaatagacaattgttaaacatgaatagaaaatattacctacttttatttttataaatacagctttagccattattataaaattcaaaagtcattttaaaaaatcaa